(19) United States
(12) Patent Application Publication
(10) Pub. No.: US 2024/0301493 A1
(43) Pub. Date: Sep. 12, 2024

(54) SYNTHETIC BARCODING OF CELL LINE BACKGROUND GENETICS

(71) Applicant: Insitro, Inc., South San Francisco, CA (US)

(72) Inventors: Max R. SALICK, San Francisco, CA (US); Eric LUBECK, San Francisco, CA (US); Srinivasan SIVANANDAN, South San Francisco, CA (US); Ajamete KAYKAS, San Francisco, CA (US)

(73) Assignee: Insitro, Inc., South San Francisco, CA (US)

(21) Appl. No.: 18/277,358

(22) PCT Filed: Feb. 17, 2022
(86) PCT No.: PCT/US2022/070707

§ 371 Date: Aug. 15, 2023

Related U.S. Application Data
(60) Provisional application No. 63/150,979, filed on Feb. 18, 2021.

(51) Int. Cl. C12Q 1/6881; C12Q 1/6841; C12Q 1/6874

(52) U.S. Cl. CPC C12Q 1/6881; C12Q 1/6841; C12Q 1/6874; C12Q 2600/136; C12Q 2600/16

(57) ABSTRACT

Provided herein are methods of pooled screening of cells from different genetic backgrounds. Also provided herein are computer-implemented methods for aligning between a first plurality of images and a second plurality of images of biological samples.

### CROSS REFERENCE TO RELATED APPLICATION

This application claims priority from U.S. Provisional Application No. 63/150,979 filed Feb. 18, 2021, entitled “SYNTHETIC BARCODING OF CELL LINE BACKGROUND GENETICS,” the contents of which are incorporated herein by reference in their entireties for all purposes.

### TECHNICAL FIELD

The present invention relates to methods of pooled screening of cells from different genetic backgrounds. The present invention also relates to computer-implemented methods for aligning between a first plurality of images and a second plurality of images.

### BACKGROUND

In cell-based models, the background genetics of a cell line can have a substantial impact on cell behavior, phenotypes, and disease states. Approaches utilizing pools of cells have the potential to enable population-based studies to be conducted in vitro. However, there are limitations to the pooled screening approaches that are currently available.

Some current pooled screening approaches depend on the background genetics of a cell as its ‘barcode’; this means that the assays may ultimately use sequencing as their final readout. This typically comes in the form of genomic DNA (gDNA) sequencing of sorted cells or as demuxlet analysis of single cell RNAseq data, enabling genotype identification based on genetic variants in the 3′ end of the single cell transcripts. This reliance on sequencing data greatly limits the number of assays that can be performed in this pooled format.

Alternative pooled approaches utilize optical barcoding. Current pooled optical barcoding and CRISPR-screening strategies generally implement a single cell line containing a variant of Cas9, along with the integration of unique padlock-flanked or otherwise sequenceable barcodes. This way, perturbing gRNAs can be identified via in situ sequencing. While a substantial technical feat, this method is capable of utilizing only a single genetic background at a time.

With the methods of pooled screening of cells from different genetic backgrounds provided herein, individual cell lines from various healthy and/or patient lines are labelled with a unique genetic barcode, which is designed to be processed via in situ sequencing after any assay. Phenotypes can then be classified and linked back to the original cell lines' genotypes, enabling statistical genetics analysis to be conducted in highly-controlled in vitro assays. This technology also allows pooled screening studies to be applied to cell lines with similar background genetics (i.e., trios and isogenically engineered lines), as well as multiplexed with perturbation-based genetic screens. Also herein are machine learning-enabled improvements of genetic barcoding methods.

The methods provided herein solve the problem of being unable to conduct large-population studies in vitro without facing difficult, costly, and artifact-sensitive scaling challenges. The pooled approach greatly reduces the effects of evaporation, temperature, and other plate-wise or well-wise artifacts that can obscure larger screens. These methods also enable population-genetics based assays to be conducted on specific cell types in a highly controlled manner, rather than on clinical outcomes which are extremely variable and affected by countless convoluting factors. Other methods, as described above, involve either sequencing-only readouts or the barcoding of the perturbagen (pooled optical barcoding), which miss the importance of non-coding variants in a given assay.

All references cited herein, including patent applications, patent publications, and scientific literature, are herein incorporated by reference in their entirety, as if each individual reference were specifically and individually indicated to be incorporated by reference.

### SUMMARY

Provided herein is a method of pooled screening of cells from different genetic backgrounds, comprising: a) labeling two or more populations of cells of different genetic backgrounds with two or more unique nucleic acid barcode sequences, each unique nucleic acid barcode sequence corresponding to a different population of cells; b) combining the two or more populations of cells to obtain a single mixed population of cells; c) performing in situ single-cell sequencing on the cells; and d) analyzing known or identifying new phenotypes of the cells in the mixed population. In some embodiments, the two or more populations of cells are from different cell lines. In some embodiments, the different cell lines are healthy cell lines. In some embodiments, the different cell lines are patient cell lines. In some embodiments, the different cell lines are isogenically engineered cell lines. In some embodiments, the different cell lines include any combination of healthy, diseased, and isogenically engineered cell lines. In some embodiments, the cells are induced pluripotent stem cells (iPSCs). In some embodiments, the iPSCs are differentiated prior to analyzing the phenotype. In some embodiments, the method further comprises culturing the cells prior to analyzing the phenotype. In some embodiments, the single mixed population of cells is on a substrate or in three-dimensional culture. In some embodiments, the substrate is a cell culture dish. In some embodiments, the method further comprises performing single cell RNAseq. In some embodiments, the method further comprises growing the two or more populations of cells for two or more generations prior to step b). In some embodiments, the method comprises stably integrating the unique nucleic acid barcode sequences into the genomes of the two or more populations of cells. In some embodiments, the unique nucleic acid barcode sequences are delivered into the cell using a virus. In some embodiments, the virus is a lentivirus. In some embodiments, the virus encodes a selectable marker. In some embodiments, the selectable marker is an antibiotic resistance gene. In some embodiments, the virus encodes a fluorescent protein. In some embodiments, each unique nucleic acid barcode sequence is at least 1 base pair in length. In some embodiments, each unique nucleic acid barcode sequence is 1 to about 18 base pairs in length. In some embodiments, each unique nucleic acid barcode sequence is 8 base pairs in length. In some embodiments, the two or more populations of cells are sequenced prior to labeling with the unique nucleic acid barcode sequences. In some embodiments, the sequencing is whole genome sequencing. In some embodiments, the two or more populations of cells were obtained from related individuals. In some embodiments, the two or more populations of cells were obtained from humans. In some embodiments, the method comprises labeling ten or more populations of cells of different genetic backgrounds with ten or more unique nucleic acid barcode sequences, each unique nucleic acid barcode sequence corresponding to a different population of cells. In some embodiments, analyzing the phenotype of the cells comprises an assay selected from the group consisting of high content imaging, calcium imaging, immunohistochemistry, cell morphology imaging, protein aggregation imaging, cell-cell interaction imaging, live cell imaging, and any other imaging-based assay modality. In some embodiments, step d) comprises analyzing the phenotype of the cells by capturing a microscopic image or a time series of microscopic images of the cells and evaluating phenotypic features presented in the image or images. In some embodiments, the method further comprises a computer-implemented technique for aligning between a first plurality of images and a second plurality of images, comprising: a) generating a first reference coordinate space of the first plurality of images, wherein the first plurality of images is of a well on a culture plate; b) extracting a first patch of the first plurality of images; c) generating a second reference coordinate space of the second plurality of images, wherein the second plurality of images is of the well on the culture plate; d) extracting a second patch of the second plurality of images; e) computing an affine transformation function between the first patch and the second patch to obtain a plurality of transformation parameters; and f) generating a coordinate transformation function between the first reference coordinate space and the second reference coordinate space based on the plurality of transformation parameters.

Also provided herein is a computer-implemented method for aligning between a first plurality of images and a second plurality of images, comprising: a) generating a first reference coordinate space of the first plurality of images, wherein the first plurality of images is of a well on a culture plate; b) extracting a first patch of the first plurality of images; c) generating a second reference coordinate space of the second plurality of images, wherein the second plurality of images is of the well on the culture plate; d) extracting a second patch of the second plurality of images; e) computing an affine transformation function between the first patch and the second patch to obtain a plurality of transformation parameters; and f) generating a coordinate transformation function between the first reference coordinate space and the second reference coordinate space based on the plurality of transformation parameters. In some embodiments, the first plurality of images is a plurality of barcoding images. In some embodiments, the second plurality of images is a plurality of marker-based/marker-free readout image. In some embodiments, the first plurality of images and the second plurality of images provide different coverage of the well. In some embodiments, the first plurality of images and the second plurality of images are taken at different times. In some embodiments, the first plurality of images and the second plurality of images have different resolutions. In some embodiments, the first plurality of images is taken by a first microscope and the second plurality of images is taken by a second microscope. In some embodiments, the first microscope is a fluorescent microscope. In some embodiments, the second microscope is a non-fluorescent microscope. In some embodiments, the first plurality of images is captured by a first imager. In some embodiments, the method further comprises detecting one or more physical characteristics of the well in the first plurality of images; and generating the first reference coordinate space based on the detected one or more physical characteristics. In some embodiments, the one or more physical characteristics of the well comprises: a shape of the well, an edge of the well, a location of the well. In some embodiments, two or more images in the first plurality of images are offset from each other by an overlap ratio, and wherein the first reference coordinate space is generated based on the overlap ratio. In some embodiments, the first reference coordinate space is generated based on metadata of the first imager. In some embodiments, the method further comprises selecting one or more marker images from the first plurality of images based on one or more landmarks captured in the one or more images, wherein the first patch is obtained from the one or more marker images from the first plurality of images. In some embodiments, the one or more landmarks comprise: one or more cells, one or more well boundaries, one or more beads, one or more nuclei, or any combination thereof. In some embodiments, the second plurality of images is captured by a second imager. In some embodiments, the method further comprises detecting one or more physical characteristics of the well in the second plurality of images; and generating the second reference coordinate space based on the detected one or more physical characteristics. In some embodiments, the one or more physical characteristics of the well comprises: a shape of the well, an edge of the well, a location of the well. In some embodiments, two or more images in the second plurality of images are offset from each other by an overlap ratio, and wherein the second reference coordinate space is generated based on the overlap ratio. In some embodiments, the second reference coordinate space is generated based on metadata of the second imager. In some embodiments, the method further comprises selecting one or more marker images from the second plurality of images based on one or more landmarks captured in the one or more images, wherein the second patch is obtained from the one or more marker images of the second plurality of images. In some embodiments, the one or more landmarks comprise: one or more cells, one or more well boundaries, one or more beads, one or more nuclei, or any combination thereof. In some embodiments, the first patch covers a center of the first image. In some embodiments, the second patch covers a center of the second image. In some embodiments, at least a portion of the first patch and at least a patch of the second patch correspond to the same subject. In some embodiments, the transformation parameters comprise one or more of: a translation parameter, a scaling parameter, and a rotation parameter. In some embodiments, the first and second images are obtained from an assay of pooled screening of cells from different genetic backgrounds, comprising: a) labeling two or more populations of cells of different genetic backgrounds with two or more unique nucleic acid barcode sequences, each unique nucleic acid barcode sequence corresponding to a different population of cells; b) combining the two or more populations of cells to obtain a single mixed population of cells; c) performing in situ single-cell sequencing on the cells; and d) analyzing known or identifying new phenotypes of the cells in the mixed population.

In some embodiments, the method further comprises utilizing a classifier configured to receive an image and output a classification result. In some embodiments, the classifier comprises a plurality of layers. In some embodiments, the classifier is a convolutional neural network. In some embodiments, the classifier is a DenseNet classifier. In some embodiments, the classification result is a classification based on the genetic background of each single cell captured in the image. In some embodiments, the method further comprises generating an embedding of the image. In some embodiments, the embedding is generated from an activation output layer prior to the last layer of the classifier. In some embodiments, the embedding is reduced in dimension using a linear dimensionality reduction method such as UMAP (Uniform Manifold Approximation and Projection for Dimension Reduction) algorithm, principal component analysis (PCA), t-distributed stochastic neighborhood embedding (t-SNE), etc. In some embodiments, the embedding is reduced in dimension using a UMAP algorithm to obtain one or more UMAP plots for visualization of the embedding. In some embodiments, the method further comprises evaluating a treatment based on at least a portion of the embedding. In some embodiments, the method further comprises evaluating a treatment based on at least a portion of the one or more UMAP plots.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an example of a vector used to introduce a unique nucleic acid barcode sequence into a cell. Features flanking the unique nucleic acid barcode sequence are shown. POSH=Pooled Optical Screening in Human cells.

FIG. 2 is a schematic showing a first modality using the method of pooled screening of cells from different genetic backgrounds described in the present application. In some embodiments, the modality includes performing an ultra-throughput drug screen. POSH=Pooled Optical Screening in Human cells.

FIG. 3 is a schematic showing a second modality using the method of pooled screening of cells from different genetic backgrounds described in the present application. In some embodiments, the modality includes performing an ultra-throughput drug screen. POSH=Pooled Optical Screening in Human cells.

FIG. 4 is a schematic showing a third modality using the method of pooled screening of cells from different genetic backgrounds described in the present application. In some embodiments, the modality includes performing an ultra-throughput drug screen.

FIG. 5 is a schematic showing the analysis pipeline used to analyze the data obtained from the method of pooled screening of cells from different genetic backgrounds described in the present application.

FIG. 6 illustrates an exemplary computer-implemented process for aligning between a first plurality of images (e.g., from a first image acquisition) and the second plurality of images (e.g., from a second image acquisition), in accordance with some embodiments.

FIG. 7 depicts an exemplary plate designed for microscopic acquisition, in accordance with some embodiments.

FIG. 8 illustrates a plurality of images of the well, in accordance with some embodiments.

FIG. 9 illustrates a plurality of images of the well, in accordance with some embodiments.

FIG. 10 illustrates two exemplary patches from two different image acquisitions, in accordance with some embodiments.

FIG. 11 illustrates an exemplary process for computing the transformation parameters, in accordance with some embodiments.

FIG. 12 illustrates an exemplary electronic device, in accordance with some embodiments.

FIGS. 13A-13B illustrate exemplary images of neuronal cells in a co-culture with astrocytes (e.g., TSC2 knockout (TSC2 ko), wild type (wt), SETD1A heterozygous knockout (SETD1A het)) stained with anti-MAP2 antibodies (FIG. 13A), and corresponding cell nuclei and cell body segmentation of neuronal single cells segmented out from background (FIG. 13B), in accordance with some embodiments.

FIG. 14A illustrates an exemplary image of genotype-labeled barcoded cells (e.g., TSC2 knockout (TSC2 ko), wild type (wt), SETD1A heterozygous knockout (SETD1A het)) captured during a round of in situ sequencing-by-synthesis, in accordance with some embodiments.

FIGS. 14B-14C illustrate exemplary counts of pooled optical screening (POSH) barcodes of each genotype (e.g., TSC2 knockout (TSC2 ko), wild type (wt), SETD1A heterozygous knockout (SETD1A het)) (FIG. 14B) and corresponding cell counts of each genotype (FIG. 14C), in accordance with some embodiments.

FIG. 15A illustrates an exemplary transformation (e.g., embedding) depicting sequencing and imaging data of untreated cells, in accordance with some embodiments. The circle with dashed lines indicates a feature embedding space predominately populated by TSC2 ko cells.

FIG. 15B illustrates an exemplary overlay of representative image patches of untreated cells over their embedding coordinates, in accordance with some embodiments.

FIG. 16A illustrates an exemplary transformation computed following treatment of cells (e.g., TSC2 knockout (TSC2 ko), wild type (wt), SETD1A heterozygous knockout (SETD1A het)) with rapamycin or without treatment (No Tr), in accordance with some embodiments. The circle with dashed lines indicates a feature embedding space predominately populated by untreated TSC2 ko cells.

FIG. 16B illustrates an exemplary transformation computed following treatment of TSC2 knockout cells with rapamycin (Rapamycin) or without treatment (No Tr), in accordance with some embodiments. The circle with dashed lines indicates a feature embedding space predominately populated by untreated TSC2 ko cells.

FIG. 17 illustrates exemplary scRNAseq embeddings following treatment of cells (e.g., TSC2 knockout (TSC2 ko), TSC2 heterozygous knockout (TSC2 het), wild type (wt), SETD1AG3 heterozygous knockout (SETD1AG3 het) SETD1AG4 heterozygous knockout (SETDAG4 het)) with various compounds (e.g., DMSO, everolimus, iadademstat, lonafarnib, rapamycin), or untreated cells. The TSC2 ko neurons appear in cluster 6, as represented by solid arrows. Clear arrows indicate the shift of all cells into a new population upon treatment with rapamycin.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The use of the alternative (e.g., “or”) should be understood to mean either one, both, or any combination thereof of the alternatives. As used herein, the indefinite articles “a” or “an” should be understood to refer to “one or more” of any recited or enumerated component.

It is understood that aspects and embodiments of the invention described herein include “comprising,” “consisting,” and “consisting essentially of” aspects and embodiments.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the term can mean up to an order of magnitude or up to 5-fold of a value. When particular values or compositions are provided in the application and claims, unless otherwise stated, the meaning of “about” should be assumed to be within an acceptable error range for that particular value or composition.

The term “optional” or “optionally” means that the subsequent described event, circumstance or substituent may or may not occur, and that the description includes instances where the event or circumstance occurs and instances where it does not.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, “isogenic” refers to organisms or cells that are characterized by essentially identical genomic DNA, for example the genomic DNA is at least about 92%, preferably at least about 98%, and most preferably at least about 99%, identical to the genomic DNA of an isogenic organism or cell.

The term “cell” is used herein in its broadest sense in the art to mean a living body that is a structural unit of tissue of a multicellular organism, surrounded by a membrane structure separated from the outside, has genetic information and has a mechanism for expression of genetic information. The cells used herein may be naturally occurring cells or artificially modified cells (for example, fused cells, genetically modified cells, etc.).

The term “differentiated cell” as used herein can refer to a cell that has been developed from an undifferentiated phenotype to a specialized phenotype. For example, embryonic cells can differentiate into epithelial cells of the intestinal lining. Differentiated cells can be isolated from, for example, fetuses or born animals.

The term “undifferentiated cell” as used herein can refer to a progenitor cell that has an undifferentiated phenotype and is capable of differentiating. An example of an undifferentiated cell is a stem cell.

As used herein, the term “stem cell” refers to a cell capable of self-renewal and pluripotency. “Pluripotent” means that a cell can give rise to the three primary germ layers, including adult animals, through its progeny; germ cells and all three germ layers, endoderm (inner gastric lining, gastrointestinal tract, lung), mesoderm (muscle, bone, blood, genitourinary), or ectoderm (epithelial tissue and nervous system). The stem cells herein may be, but are not limited to, embryonic stem (ES) cells, tissue stem cells (also referred to as tissue-specific stem cells or somatic stem cells), or induced pluripotent stem cells. Artificially produced cells (e.g., reprogrammed cells) having the above-described capabilities may be stem cells.

The term “embryonic stem (ES) cell” as used herein can refer to a pluripotent cell isolated from an embryo maintained in an in vitro cell culture medium.

Tissue stem cells are divided into categories based on the site from which the cells are derived. For example, the skin system (e.g., epidermal stem cells, hair follicle stem cells), digestive system (e.g., pancreatic stem cells, liver stem cells, etc.), bone marrow (e.g., hematopoietic stem cells, mesenchymal stem cells, etc.) and nervous systems (e.g., neural stem cells, retinal stem cells, etc.).

“Induced pluripotent stem cells”, generally abbreviated as iPS cells or iPSCs, are pluripotent stem cells derived from non-pluripotent cells, typically adult somatic cells, or terminally differentiated cells such as fibroblasts, hematopoietic cells, muscle cells, or nerve cells. This refers to a type of pluripotent stem cell that has been artificially prepared by expressing reprogramming factors from temporarily differentiated cells such as epithelial cells.

“Self-renewal” refers to the ability to go through many cycles of cell division while maintaining an undifferentiated state.

As used herein, the term “somatic cell” refers to any cell other than a germ cell, such as an egg or sperm. Typically, somatic cells have limited or no pluripotency. As used herein, somatic cells may be natural or genetically modified.

“Single cell RNAseq” or “scRNA-Seq,” as used herein, generally refers to a single-cell RNA sequencing method to obtain expression profiles of individual cells.

The term “Whole Genome Sequencing (WGS)” herein refers to a process whereby the sequence of the entire genome of an organism, for example, humans, dogs, mice, viruses or bacteria can be determined. It is not necessary that the entire genome actually be sequenced.

The term “sequencing” herein refers to a method for determining the nucleotide sequence of a polynucleotide e.g. genomic DNA. Preferably, sequencing methods include as non-limiting examples next generation sequencing (NGS) methods, (NGS) in which clonally amplified DNA templates or single DNA molecules are sequenced in a massively parallel fashion (e.g. as described in Volkerding et al. Clin Chem 55:641-658 (2009); Metzker M Nature Rev 11:31-46 (2010)).

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Methods of the Invention

One aspect of the present invention provides a method of pooled screening of cells from different genetic backgrounds, comprising: a) labeling two or more populations of cells of different genetic backgrounds with two or more unique nucleic acid barcode sequences, each unique nucleic acid barcode sequence corresponding to a different population of cells; b) combining the two or more populations of cells to obtain a single mixed population of cells; c) performing in situ single-cell sequencing on the cells; and d) analyzing known or identifying new phenotypes of the cells in the mixed population. In some embodiments, the two or more populations of cells include any combination of cultured cells, primary cells, post-mitotic cells (such as neural cells), and tissue sections. In some embodiments, the two or more populations of cells are from different cell lines. In some embodiments, the different cell lines are healthy cell lines. In some embodiments, the different cell lines are patient cell lines. In some embodiments, the different cell lines are isogenically engineered cell lines. In some embodiments, the different cell lines include any combination of healthy, patient, and isogenically engineered cell lines. In some embodiments, the two or more populations of cells are induced pluripotent stem cells (iPSCs). In some embodiments, the method further comprises culturing the cells prior to analyzing the phenotype. In some embodiments, the two or more populations of cells were obtained from humans. In some embodiments, step d) comprises analyzing the phenotype of the cells by capturing a microscopic image or a time series of microscopic images of the cells and evaluating phenotypic features presented in the image or images. In some embodiments, the method further comprises a computer-implemented technique for aligning between a first plurality of images and a second plurality of images, comprising: 1) generating a first reference coordinate space of the first plurality of images, wherein the first plurality of images is of a well on a culture plate; 2) extracting a first patch of the first plurality of images; 3) generating a second reference coordinate space of the second plurality of images, wherein the second plurality of images is of the well on the culture plate; 4) extracting a second patch of the second plurality of images; 5) computing an affine transformation function between the first patch and the second patch to obtain a plurality of transformation parameters; and 6) generating a coordinate transformation function between the first reference coordinate space and the second reference coordinate space based on the plurality of transformation parameters.

In some embodiments, the method further comprises utilizing a classifier configured to receive an image, such as an aligned imaged generated from a first plurality of images and a second plurality of images according to the methods provided herein, and output a classification result. The classifier can be a neural network comprising a plurality of layers. In some embodiments, the classifier is a convolutional neural network (CNN), e.g., a DenseNet classifier. In some embodiments, the classifier can be used to obtain low-dimensional representations of an image, such as an embedding. An embedding can be generated from a layer of the plurality of layers of the classifier. For example, an activation output layer prior to the last layer of the classifier can be utilized as low-dimensional representations of an image for visualization. In some embodiments, the embedding is reduced in dimension using a dimensionality reduction method. In some embodiments, the dimensionality reduction method is an unsupervised linear dimensionality reduction method. In some embodiments, the dimensionality reduction method is an unsupervised non-linear dimensionality reduction method. In some embodiments, the embedding is reduced in dimension using the UMAP (Uniform Manifold Approximation and Projection for Dimension Reduction) algorithm, principal component analysis (PCA), t-distributed stochastic neighborhood embedding (t-SNE), or any other suitable dimensionality reduction methods. In some embodiments, the embedding is reduced in dimension using the UMAP algorithm to obtain UMAP plots for visualization of the embedding.

In some embodiments, the method further comprises growing the two or more populations of cells for two or more generations prior to step b). In some embodiments, the method further comprises growing the two or more populations of cells for 3 or more generations, 4 or more generations, 5 or more generations, 6 or more generations, 7 or more generations, 8 or more generations, 9 or more generations, or 10 or more generations prior to step b).

In some embodiments, performing in situ single-cell sequencing comprises using fluorescent in situ RNA sequencing (FISSEQ) (Lee et al., Nature Protocols 2015, 10(3):442-58). In this method, mRNAs are reverse transcribed in situ using aminoallyl dUTP and adapter sequence-tagged random hexamers. The resulting cDNA fragments are fixed to the cellular protein matrix and circularized. The circular templates are amplified by rolling circle amplification (RCA) followed by sequencing and imaging. This method allows for simultaneous detection of tissue-specific gene expression, RNA splicing, post-transcriptional modifications, and preservation of their spatial information. It is a relatively unbiased method and can achieve transcriptome-wide sampling.

In some embodiments, performing in situ single-cell sequencing comprises using padlock in situ sequencing method (Ke et al. Nature Methods 2013, 10(9)857-60). In this method, after mRNA is reverse transcribed into cDNA, the mRNA is degraded by RNaseH. A padlock probe then binds to the cDNA with a gap between the probe ends over the bases targeted for sequencing. The gap is filled by DNA polymerization and ligated to form a circularized molecule. The circular templates are amplified by RCA followed by sequencing and imaging. Similar to FISSEQ, padlock in situ sequencing allows for preservation of spatial information of analyzed RNA sequences.

In some embodiments, following RCA, the sequencing of the amplified DNA can be achieved using sequencing by ligation or sequencing by synthesis. Sequencing by synthesis relies on a DNA polymerase to incorporate four reversible terminator-bound dNTPs. One base is added per cycle and the fluorescently labeled reversible terminator is imaged as each dNTP is added. Sequencing by ligation uses the mismatch sensitivity of DNA ligase instead to distinguish the sequence of interest and incorporate a pool of fluorescently labeled oligonucleotides of varying lengths. Sequencing by ligation has high accuracy but may encounter problems with palindromic sequences.

In some embodiments, rather than in situ sequencing of RNA, the DNA barcode is directly read via methods such as peptidic nucleic acids, locked nucleic acids, transposases, Zombie, or other in situ transcription methods.

In some embodiments, rather than in situ sequencing of RNA or DNA, protein tags are used as the barcode to label individual cell lines, using methods such as ProCodes.

In certain embodiments, multiple barcode sequences within the same cell can be determined by in situ sequencing. The barcode screening method can also be combined with high-dimensional morphological profiling and in situ multiplexed gene expression analysis. In certain embodiments, phenotypes can be measured within the native spatial context using in situ sequencing of tissue samples.

In some embodiments, the method further comprises performing single cell RNAseq (scRNA-seq). In some embodiments, a single unique nucleic acid barcode sequence is used for in situ single-cell sequencing and scRNA-seq. scRNA-seq approaches include 10× Genomics, Drop-seq, and Seq-well, inDrops, Rhapsody, and Split-Seq. For example, single-cell libraries can be prepared from single-cell suspensions using Chromium with v2 chemistry (10× Genomics). Such single-cell libraries can be sequenced (e.g., a NextSeq 500 (Illumina)). Sequencing reads may be processed, for example, by alignment, filtration, de-duplication, and/or conversion into a digital count matrix using Cell Ranger 1.2 (10× Genomics).

In some embodiments, the method comprises the application of sc-RNAseq and utilization of a compressed sensing methodology for RNA sequencing. Examples include L1000 and hybrid capture

In some embodiments, the method further comprises culturing the single mixed population of cells prior to analyzing the phenotype. In some embodiments, the culture medium used to culture the cells contains serum. In some embodiments, the culture medium used to culture the cells is serum-free. A serum-free medium refers to a medium that does not contain untreated or unpurified serum, and thus can include a medium having purified blood-derived components or animal tissue-derived components (e.g., growth factors). From the viewpoint of preventing contamination with different animal-derived components, the serum may be derived from the same animal as the cells. The culture medium may or may not contain a serum replacement. Serum substitutes include albumin (albumin substitutes such as lipid-rich albumin, recombinant albumin, plant starch, dextran and protein hydrolysates), transferrin (or other iron transporters), fatty acids, insulin, collagen precursors, trace elements, 2-mercaptoethanol, or 3′-thioglycerol, or an equivalent thereof.

In some embodiments, the single mixed population of cells is on a substrate or in a three dimensional culture. In some embodiments, the mixed population of cells is on a substrate. In some embodiments, the substrate is any standard tissue culture container such as a tissue culture plate or flask. In some embodiments, the substrate is a cell culture dish. In some embodiments, the substrate is a tissue culture plate. In some embodiments, the substrate is a petri dish. In some embodiments, the substrate is a tissue culture flask. In some embodiments, the substrate is a well of a standard microwell plate, such as a 6-well, 12-well, 24-well, 96-well, 384-well, or 1,536-well plate. In some embodiments, the substrate is a 6-well plate. In some embodiments, the substrate is a 12-well plate. In some embodiments, the substrate is a 24-well plate. In some embodiments, the substrate is a 96-well plate. In some embodiments, the substrate is a 384-well plate. In some embodiments, the substrate is a 1,536-well plate. The substrate may be made of any material for imaging using the imaging modalities described herein. In certain embodiments, the plate may be plastic-bottom plates suitable for imaging using the imaging modalities described herein. In certain embodiments, the plate may be glass-bottom plates suitable for imaging using the imaging modalities described herein. In certain embodiments, the substrate may be a culture chamber in an array of culture chambers defined on a microfluidic device, or droplet generated on a microfluidic device. In certain example embodiments, a single cell or population of cells may be cultured on individual microscopic slides in culture medium. In some embodiments, the single mixed population of cells is in three-dimensional culture. In some embodiments, the three-dimensional culture comprises a scaffold. In some embodiments, the scaffold comprises a three-dimensional matrix. In some embodiments, the three-dimensional matrix comprises a material selected from the group consisting of BD Matrigel™ basement membrane matrix (BD Sciences), Cultrex® basement membrane extract (BME; Trevigen), hyaluronic acid, polyethylene glycol (PEG), polyvinyl alcohol (PVA), polylactide-co-glycolide (PLG), and polycaprolactone (PLA). In some embodiments, the three-dimensional matrix comprises BD Matrigel™ basement membrane matrix (BD Sciences). In some embodiments, the three-dimensional matrix comprises Cultrex® basement membrane extract (BME; Trevigen). In some embodiments, the three-dimensional matrix comprises hyaluronic acid. In some embodiments, the three-dimensional matrix comprises polyethylene glycol (PEG). In some embodiments, the three-dimensional matrix comprises polyvinyl alcohol (PVA). In some embodiments, the three-dimensional matrix comprises polylactide-co-glycolide (PLG). In some embodiments, the three-dimensional matrix comprises polycaprolactone (PLA). In some embodiments, the three-dimensional culture is scaffold-free.

In some embodiments, the method comprises stably integrating the unique nucleic acid barcode sequences into the genomes of the two or more populations of cells. In some embodiments, the unique nucleic acid barcode sequences are delivered into the cell using a virus. In some embodiments, the virus is a retrovirus. In some embodiments, the retrovirus is or is derived from a Moloney murine leukemia virus (MMULV), feline immunodeficiency virus (FIV), Harvey murine sarcoma virus (HaMuSV), murine mammary tumor virus (MuMTV), gibbon ape leukemia virus (GaLV), human immunodeficiency virus (HIV), Rous Sarcoma Virus (RSV), or lentivirus. In some embodiments, the virus is a lentivirus. In some embodiments, the virus is derived from a lentivirus. In some embodiments, the U3 sequence from the lentiviral 5′ LTR may be replaced with a promoter sequence in the viral construct. This may increase the titer of virus recovered from the packaging cell line. An enhancer sequence may also be included. In some embodiments, the virus encodes a selectable marker. In some embodiments, the selectable marker is an antibiotic resistance gene. In some embodiments, the antibiotic resistance gene confers resistance to an antibiotic selected from the group consisting of puromycin, hygromycin, bleomycin, neomycin, actinomycin D, and mitomycin C. In some embodiments, the antibiotic resistance gene confers resistance to puromycin. In some embodiments, the antibiotic resistance gene confers resistance to hygromycin. In some embodiments, the antibiotic resistance gene confers resistance to bleomycin. In some embodiments, the antibiotic resistance gene confers resistance to neomycin. In some embodiments, the antibiotic resistance gene confers resistance to actinomycin D. In some embodiments, the antibiotic resistance gene confers resistance to mitomycin C. In some embodiments, the virus encodes one or more fragments of antibiotic resistance genes. In some embodiments, the virus encodes a fluorescent protein. In some embodiments, the fluorescent protein is selected from the group consisting of a green fluorescent protein, a red fluorescent protein, a blue fluorescent protein, a cyan fluorescent protein, a yellow fluorescent protein, and an orange fluorescent protein.

In some embodiments, the unique nucleic acid barcode sequence is a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment.

The unique nucleic acid barcode sequence can be attached, or “tagged,” to a target molecule. This attachment can be direct (for example, covalent or noncovalent binding of the unique nucleic acid barcode sequence to the target molecule) or indirect (for example, via an additional molecule).

Target molecules can be optionally labeled with multiple unique nucleic acid barcode sequences in combinatorial fashion (for example, using multiple unique nucleic acid barcode sequences bound to one or more specific binding agents that specifically recognizing the target molecule), thus greatly expanding the number of unique identifiers possible within a particular unique nucleic acid barcode sequence pool. In certain embodiments, unique nucleic acid barcode sequences are added to a growing barcode concatemer attached to a target molecule, for example, one at a time. In other embodiments, multiple unique nucleic acid barcode sequences are assembled prior to attachment to a target molecule.

In some embodiments, each unique nucleic acid barcode sequence is at least 1 base pair in length. In some embodiments, each unique nucleic acid barcode sequence is 1 to about 18 base pairs in length. In some embodiments, each unique nucleic acid barcode sequence is 1 to about 12 base pairs in length. In some embodiments, each unique nucleic acid barcode sequence is 1 to about 10 base pairs in length. In some embodiments, each unique nucleic acid barcode sequence is 1 to about 8 base pairs in length. In some embodiments, each unique nucleic acid barcode sequence is 2 to about 12 base pairs in length. In some embodiments, each unique nucleic acid barcode sequence is 2 to about 10 base pairs in length. In some embodiments, each unique nucleic acid barcode sequence is 2 to about 8 base pairs in length. In some embodiments, each unique nucleic acid barcode sequence is 3 to about 12 base pairs in length. In some embodiments, each unique nucleic acid barcode sequence is 3 to about 10 base pairs in length. In some embodiments, each unique nucleic acid barcode sequence is 3 to about 8 base pairs in length. In some embodiments, each unique nucleic acid barcode sequence is 4 to about 12 base pairs in length. In some embodiments, each unique nucleic acid barcode sequence is 4 to about 10 base pairs in length. In some embodiments, each unique nucleic acid barcode sequence is 4 to about 8 base pairs in length. In some embodiments, each unique nucleic acid barcode sequence is 5 to about 12 base pairs in length. In some embodiments, each unique nucleic acid barcode sequence is 5 to about 10 base pairs in length. In some embodiments, each unique nucleic acid barcode sequence is 5 to about 8 base pairs in length. In some embodiments, each unique nucleic acid barcode sequence is 6 to about 12 base pairs in length. In some embodiments, each unique nucleic acid barcode sequence is 6 to about 10 base pairs in length. In some embodiments, each unique nucleic acid barcode sequence is 6 to about 8 base pairs in length. In some embodiments, each unique nucleic acid barcode sequence is 1 base pair in length. In some embodiments, each unique nucleic acid barcode sequence is 2 base pairs in length. In some embodiments, each unique nucleic acid barcode sequence is 3 base pairs in length. In some embodiments, each unique nucleic acid barcode sequence is 4 base pairs in length. In some embodiments, each unique nucleic acid barcode sequence is 5 base pairs in length. In some embodiments, each unique nucleic acid barcode sequence is 6 base pairs in length. In some embodiments, each unique nucleic acid barcode sequence is 7 base pairs in length. In some embodiments, each unique nucleic acid barcode sequence is 8 base pairs in length. In some embodiments, each unique nucleic acid barcode sequence is 9 base pairs in length. In some embodiments, each unique nucleic acid barcode sequence is 10 base pairs in length. In some embodiments, each unique nucleic acid barcode sequence is 11 base pairs in length. In some embodiments, each unique nucleic acid barcode sequence is 12 base pairs in length. In some embodiments, each unique nucleic acid barcode sequence is 13 base pairs in length. In some embodiments, each unique nucleic acid barcode sequence is 14 base pairs in length. In some embodiments, each unique nucleic acid barcode sequence is 15 base pairs in length. In some embodiments, each unique nucleic acid barcode sequence is 16 base pairs in length. In some embodiments, each unique nucleic acid barcode sequence is 17 base pairs in length. In some embodiments, each unique nucleic acid barcode sequence is 18 base pairs in length. In certain embodiments, the unique nucleic acid barcode sequence may be detected directly using an in situ sequencing method. In certain example embodiments, the unique nucleic acid barcode sequence is detected using fluorescent in situ RNA sequencing (FISSEQ), in situ mRNA-seq, padlock in situ sequencing, sequencing by ligation, SOLID® sequencing, sequencing by synthesis, peptidic nucleic acids, locked nucleic acids, transposases, Zombie, other in situ transcription methods, or other protein-based or peptide-based barcoding technologies such as ProCodes. In certain example embodiments, the mRNA transcript encoding the unique nucleic acid barcode sequence is sequenced. In certain other example embodiments, a cDNA copy of the mRNA is first generated and then sequenced. In certain other example embodiments, the DNA containing the barcode is directly sequenced.

In some embodiments, the two or more populations of cells are sequenced prior to labeling with the unique nucleic acid barcode sequence. In some embodiments, portions of the genomes of the two or more populations of cells are sequenced. In some embodiments the sequencing is whole genome sequencing. In some embodiments, the whole genome sequencing comprises the use of next generation sequencing (NGS). NGS technologies for determining the entire human genome sequence have been previously described (Levy et al PLOS Biol 55, 254 (2007), Wheeler et al Nature 452:872-876 (2008): Bentley et al., Nature 456:53-59 (2008).

In some embodiments, the two or more populations of cells are obtained from related individuals. In some embodiments, the related individuals are parents and offspring. In some embodiments, the related individuals are siblings. In some embodiments, the two or more populations of cells are obtained from individuals with low genetic diversity. In some embodiments, the two or more populations of cells are obtained from individuals with high genetic diversity. In some embodiments, the two or more populations of cells are comprised from a combination of groups containing low and high genetic diversity.

In some embodiments, the method comprises labeling three or more populations of cells of different genetic backgrounds with three or more unique nucleic acid barcode sequences, each unique nucleic acid barcode sequence corresponding to a different population of cells. In some embodiments, the method comprises labeling four or more populations of cells of different genetic backgrounds with four or more unique nucleic acid barcode sequences, each unique nucleic acid barcode sequence corresponding to a different population of cells. In some embodiments, the method comprises labeling five or more populations of cells of different genetic backgrounds with five or more unique nucleic acid barcode sequences, each unique nucleic acid barcode sequence corresponding to a different population of cells. In some embodiments, the method comprises labeling six or more populations of cells of different genetic backgrounds with six or more unique nucleic acid barcode sequences, each unique nucleic acid barcode sequence corresponding to a different population of cells. In some embodiments, the method comprises labeling seven or more populations of cells of different genetic backgrounds with seven or more unique nucleic acid barcode sequences, each unique nucleic acid barcode sequence corresponding to a different population of cells. In some embodiments, the method comprises labeling eight or more populations of cells of different genetic backgrounds with eight or more unique nucleic acid barcode sequences, each unique nucleic acid barcode sequence corresponding to a different population of cells. In some embodiments, the method comprises labeling nine or more populations of cells of different genetic backgrounds with nine or more unique nucleic acid barcode sequences, each unique nucleic acid barcode sequence corresponding to a different population of cells. In some embodiments, the method comprises labeling ten or more populations of cells of different genetic backgrounds with ten or more unique nucleic acid barcode sequences, each unique nucleic acid barcode sequence corresponding to a different population of cells. In some embodiments, the method comprises labeling 11 or more populations of cells of different genetic backgrounds with 11 or more unique nucleic acid barcode sequences, each unique nucleic acid barcode sequence corresponding to a different population of cells. In some embodiments, the method comprises labeling 12 or more populations of cells of different genetic backgrounds with 12 or more unique nucleic acid barcode sequences, each unique nucleic acid barcode sequence corresponding to a different population of cells. In some embodiments, the method comprises labeling 13 or more populations of cells of different genetic backgrounds with 13 or more unique nucleic acid barcode sequences, each unique nucleic acid barcode sequence corresponding to a different population of cells. In some embodiments, the method comprises labeling 14 or more populations of cells of different genetic backgrounds with 14 or more unique nucleic acid barcode sequences, each unique nucleic acid barcode sequence corresponding to a different population of cells. In some embodiments, the method comprises labeling 15 or more populations of cells of different genetic backgrounds with 15 or more unique nucleic acid barcode sequences, each unique nucleic acid barcode sequence corresponding to a different population of cells. In some embodiments, the method comprises labeling 20 or more populations of cells of different genetic backgrounds with 20 or more unique nucleic acid barcode sequences, each unique nucleic acid barcode sequence corresponding to a different population of cells. In some embodiments, the method comprises labeling 50 or more populations of cells of different genetic backgrounds with 50 or more unique nucleic acid barcode sequences, each unique nucleic acid barcode sequence corresponding to a different population of cells. In some embodiments, the method comprises labeling 100 or more populations of cells of different genetic backgrounds with 100 or more unique nucleic acid barcode sequences, each unique nucleic acid barcode sequence corresponding to a different population of cells. In some embodiments, the method comprises labeling 500 or more populations of cells of different genetic backgrounds with 500 or more unique nucleic acid barcode sequences, each unique nucleic acid barcode sequence corresponding to a different population of cells. In some embodiments, the method comprises labeling 1000 or more populations of cells of different genetic backgrounds with 1000 or more unique nucleic acid barcode sequences, each unique nucleic acid barcode sequence corresponding to a different population of cells. In some embodiments, the method comprises labeling 2000 or more populations of cells of different genetic backgrounds with 2000 or more unique nucleic acid barcode sequences, each unique nucleic acid barcode sequence corresponding to a different population of cells. In some embodiments, the method comprises labeling 5000 or more populations of cells of different genetic backgrounds with 5000 or more unique nucleic acid barcode sequences, each unique nucleic acid barcode sequence corresponding to a different population of cells. In some embodiments, the method comprises labeling 10,000 or more populations of cells of different genetic backgrounds with 10,000 or more unique nucleic acid barcode sequences, each unique nucleic acid barcode sequence corresponding to a different population of cells. In some embodiments, the method comprises labeling up to two orders of magnitude above 100 populations of cells of different genetic backgrounds with two orders of magnitude above 100 unique nucleic acid barcode sequences, each unique nucleic acid barcode sequence corresponding to a different population of cells.

In some embodiments, analyzing the phenotype of the single mixed population of cells comprises an assay selected from the group consisting of high content imaging, calcium imaging, immunohistochemistry, cell morphology imaging, protein aggregation imaging, cell-cell interaction imaging, live cell imaging, and any other imaging-based assay modality. In some embodiments, analyzing the phenotype of the single mixed population of cells comprises an assay selected from the group consisting of high content imaging, calcium imaging, immunohistochemistry, cell morphology imaging, protein aggregation imaging, cell-cell interaction imaging, and live cell imaging. In some embodiments, analyzing the phenotype of the single mixed population of cells comprises performing high content imaging. In some embodiments, analyzing the phenotype of the single mixed population of cells comprises performing calcium imaging. In some embodiments, analyzing the phenotype of the single mixed population of cells comprises performing immunohistochemistry. In some embodiments, analyzing the phenotype of the single mixed population of cells comprises performing cell morphology imaging. In some embodiments, analyzing the phenotype of the single mixed population of cells comprises performing protein aggregation imaging. In some embodiments, analyzing the phenotype of the single mixed population of cells comprises performing cell-cell interaction imaging. In some embodiments, analyzing the phenotype of the single mixed population of cells comprises performing live cell imaging.

### Stem Cells

In some embodiments, the cells are stem cells. In some embodiments, the stem cells are pluripotent stem cells. In some embodiments, the cells are induced pluripotent stem cells (iPSCs). In some embodiments, iPSCs are generated from somatic cells by introducing one or more known reprogramming factors. In some embodiments, the iPSCs are differentiated prior to analyzing the phenotype. Stem cells are characterized by their ability to renew themselves through division of mitotic cells and differentiation into a diverse range of specialized cell types. There are two major types of mammalian stem cells: embryonic stem cells found in blastocysts and adult stem cells found in adult tissues. In developing embryos, stem cells can differentiate into any specialized embryonic tissue. In adults, stem and progenitor cells function as a repair system for the body, not only recruiting specialized cells, but also maintaining normal turnover of regenerative organs such as blood, skin, or intestinal tissue. Human embryonic stem cells (hES) can be defined by the presence of several transcription factors and cell surface proteins. Transcription factors Oct4, Nanog, and Sox2 form a core regulatory network that reliably represses genes that lead to the maintenance of differentiation and pluripotency. The cell surface antigens most often used to identify hES cells include glycolipids SSEA3 and SSEA4 and keratan sulfate antigens Tra-1-60 and Tra-1-81.

The generation of iPSCs depends on the gene or genes used for induction. Factors such as Oct3/4, KLF4, Sox2 and/or c-myc or combinations thereof can be used. Nucleic acids encoding these reprogramming factors can be included in monocistronic or multicistronic expression cassettes. Similarly, the nucleic acid encoding the monocistronic or multicistronic expression cassette can be included in one reprogramming vector or multiple reprogramming vectors.

iPSCs are typically generated by transfecting specific stem cell-related genes into non-pluripotent cells such as adult fibroblasts or cord blood cells. Transfection can be accomplished with integrated viral vectors such as retroviruses (e.g., lentiviruses) or non-integrated viral vectors such as Sendai virus. Reprogramming may also be done using virus-free methods such as episomal reprogramming or mRNA reprogramming. After the critical period, a small number of transfected cells begin to resemble morphologically and biochemically to pluripotent stem cells can be separated based on morphological selection, doubling time, reporter gene expression, and/or antibiotic resistance.

Pluripotent cells can be cultured and maintained in an undifferentiated state using various methods. In some embodiments, matrix components may be included in a given medium to culture and maintain pluripotent cells in a substantially or essentially undifferentiated state. Various matrix components can be used to culture and maintain pluripotent cells such as hESCs or iPSCs. For example, collagen IV, fibronectin, laminin, and vitronectin may be used in combination to provide a solid support for embryonic cell culture and maintenance.

Matrigel™ may be used to provide a substrate for cell culture and maintenance of pluripotent cells. Matrigel™ is a gelatinous protein mixture secreted by mouse tumor cells and is commercially available from BD Biosciences (New Jersey, USA). The mixture resembles the complex extracellular environment found in many tissues and is used by cell biologists as a substrate for cell culture. It will be appreciated that additional methods of culturing and maintaining iPSCs are well known to those of skill in the art and may be used with embodiments of the present invention.

In some embodiments, the method comprises analyzing the differentiation of the stem cells. In some embodiments, the method comprises analyzing the differentiation of the pluripotent stem cells. In some embodiments, the method comprises analyzing the differentiation of the iPSCs. In some embodiments, the method comprises analyzing the cell types derived from stem cell differentiation.

### Microscopic Image Registration

In some embodiments, an exemplary platform includes microscopic image registration techniques. The techniques can be used to obtain a transformation between sequencing and marker-based/marker-free readouts for demultiplexing single-cell image based readouts.

Image registration is the process of transforming different sets of images into one coordinate system. The different sets of images may be of the same object(s) but from different image acquisitions—in other words, the different sets of images may be taken by different imagers (e.g., different microscopes) at different times using different settings and thus may have different depths, different resolutions, different viewpoints, etc. For example, each base of the barcode can be captured as an independent acquisition spaced out in time with potential physical movement between acquisitions. Further, the marker/phenotype acquisition can be captured at a different resolution, time, and/or from a different microscope compared to the barcode image acquisitions.

Existing methods for registration between microscopic image acquisitions are performed either at the field level or at the well level, and both suffer from deficiencies. For example, field-level registration cannot be used for acquisition taken at different settings or between different microscopes, and movement of the plate between the acquisition can lead to loss of information. Well-level registration is computationally expensive and is not scalable for larger well sizes and higher magnifications as the complexity of the alignment method is O(N log N) where N is the number of pixels in the image. The deficiencies of current approaches are described in detail herein.

The image registration techniques in the present disclosure involves computing a transformation function (e.g., a fixed affine transformation function) between two acquisitions. The two acquisitions refer to two sets of images taken using two different acquisition settings, at two different time points, and/or using two different imagers (e.g., different microscopes). After computing such a transformation function, obtaining overlapping information can be done by applying the transformation to the coordinates of the field-of-view images in source acquisition independently to obtain the corresponding field-of-view images and coordinates in the target acquisition.

Thus, the image registration techniques in the present disclosure can be used to obtain a transformation of coordinates between two different acquisitions of the same object(s), wherein the two acquisitions may have occurred using different settings and different equipment and potential human interactions may have occurred between the acquisitions. The techniques can be useful in a variety of laboratory image-based experimental settings.

For example, embodiments of the present disclosure can be used to perform alignment of physical coordinates between images acquired from two different microscopes (fluorescent/non-fluorescent). For example, one image may be from a microscope inside an incubator in a live imaging setup while the other being a fluorescence image acquired after fixation. As another example, embodiments of the present disclosure can be used to perform alignment of physical coordinates between images acquired at two different resolutions for multi-scale image analysis and reconstruction. As another example, embodiments of the present disclosure can be used to perform alignment of physical coordinates between images obtained from a fixed sample after consecutive washing and/or multiple staining procedures involving movement of plates between the microscope and automation setup, for example, sequencing by synthesis cycles for barcoding cells in pooled optical screening analysis.

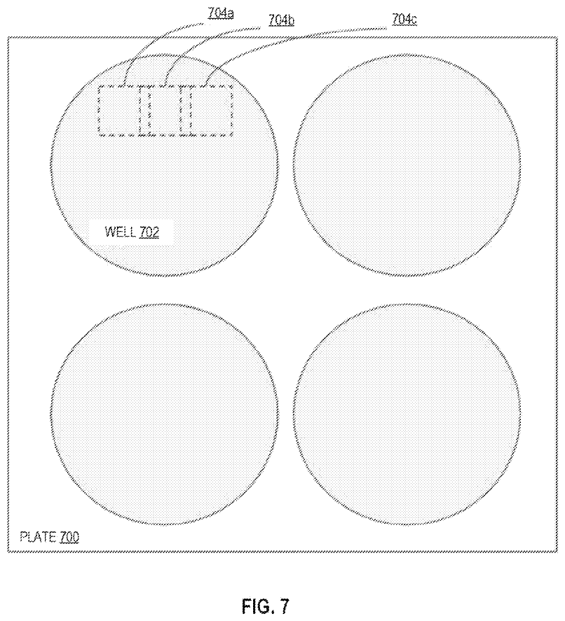

Microscopic acquisitions for high-content, high-throughput imaging generally involves imaging cells that are cultured in a special-purpose plastic/glass bottom plate. FIG. 7 depicts an exemplary plate 700 designed for microscopic acquisition, in accordance with some embodiments. The plate 700 includes a plurality of wells (e.g., 6, 24, 86, 384) wells, such as well 702. Each of the plurality of wells contains cells treated with predefined condition(s) according to the experimental design. In some embodiments, multiple wells on the plate are treated with the same condition(s); in some embodiments, the wells are treated with different condition(s).

With reference to FIG. 7, a well is generally imaged in parts as a collection of overlapping fields of view images by a microscopic camera. For example, a typical microscopic camera has an image output size of approximately 2,000×2,000 pixels. The resolution of the images can vary based on the magnification of the objective used for imaging. As shown in FIG. 7, three separate field-of-view images 704a, 704b, and 704c can be taken of the well 702.

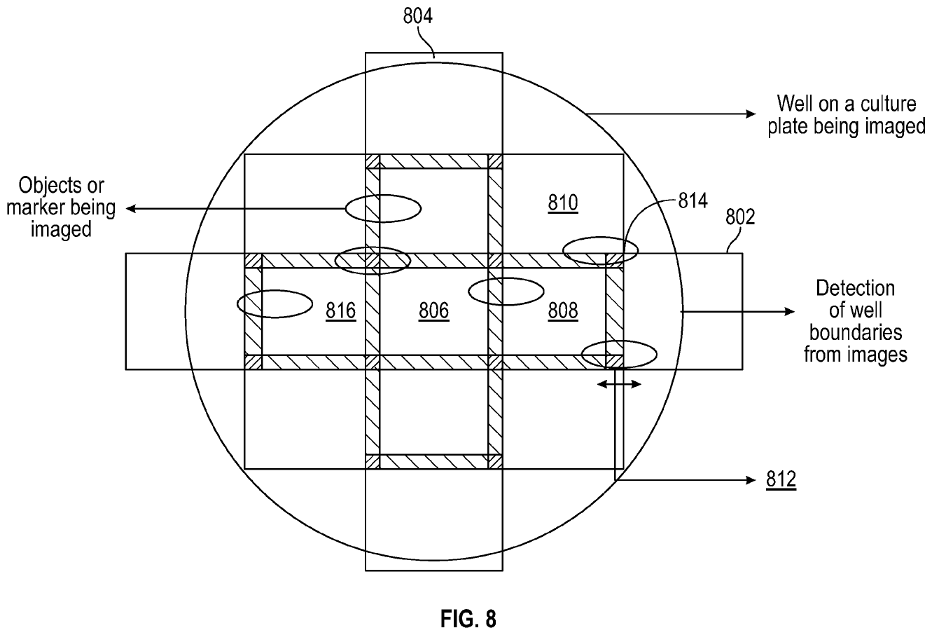

FIG. 8 illustrates a plurality of images of the well, in accordance with some embodiments. As shown, the plurality of images include images capturing the boundaries of the well (e.g., images 802 and 804) and the center of the well (e.g., image 806). The plurality of images are of the same size, and neighboring images overlap with each other. For example, images 802 and 808 overlap with each other horizontally by 812. As another example, images 802, 808, and 810 overlap with each other by area 814.

Performing image registration for microscopic image acquisitions can be challenging for a number of reasons. Image registration is the process of transforming different sets of images into one coordinate system. The two different sets of images (i.e., two image acquisitions) may be from different sensors (e.g., different microscopes), different times, different depths, different resolutions, and/or different viewpoints. Existing methods for registration between microscopic image acquisitions are performed either at the field level or well level, and both suffer from deficiencies as described below.

Current field-level registration involves establishing one-to-one correspondence between field-of-view images acquired from two image acquisitions. One-to-one correspondence is established by the order of image acquisition within a field and field of view position in the well. Accordingly, a field of view image captured in the first acquisition is registered with the same field of view image captured in the second acquisition. However, field-level registration only works for acquisitions taken using the same settings and the same microscope. It cannot be used for acquisition taken at different settings or between different microscopes, and movement of the plate between the acquisition can lead to loss of information.

Current well-level registration involves reconstruction of a well image by stitching smaller field of view images and aligning the well images using a Fourier correlation based or related methodology. Although this method can work for smaller well sizes and lower objective magnifications (as the well image size in terms of number of pixels is smaller), it is not scalable for larger well sizes and higher magnifications as the complexity of the alignment method is O(N log N) where N is the number of pixels in the image.

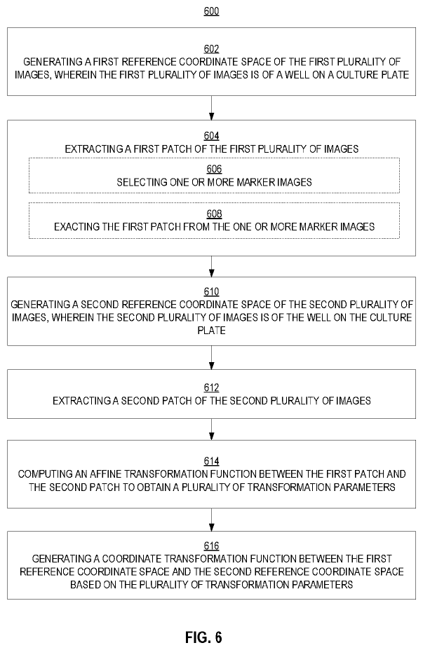

FIG. 6 illustrates an exemplary computer-implemented process 600 for aligning between a first plurality of images (e.g., from a first image acquisition) and a second plurality of images (e.g., from a second image acquisition). The first and second acquisitions may be different in terms of coverage of the well, image resolution, image size, and imager type, and the relative position of the well may have been shifted/rotated, but the underlying object(s) being imaged are the same. The process 600 can be performed at least in part using one or more electronic devices. In some embodiments, the blocks of 600 can be divided up between multiple electronic devices. Some blocks can be optionally combined, the order of some blocks can be optionally changed, and some blocks are optionally omitted. In some examples, additional steps may be performed in combination with the process. Accordingly, the operations as illustrated are exemplary by nature and, as such, should not be viewed as limiting.

In the exemplary process 600, the first plurality of images and the second plurality of images are both of a well (e.g., well 702) on a culture plate, but they are from two different image acquisitions. While the content in the well remains the same between the two image acquisitions, the imaging settings may have changed between the two image acquisitions. For example, the first plurality of images may be captured using a first imager (e.g., a fluorescent microscope) and the second plurality of images may be captured using a second imager (e.g., a non-fluorescent microscope). As another example, the first plurality of images may have a different resolution than the second plurality of images. As another example, the first plurality of images and the second plurality of images provide different coverage of the well (e.g., the second coverage may be of a different size and/or may be shifted compared to the first coverage). As another example, the first plurality of images and the second plurality of images are taken at different times.

In some embodiments, the images are obtained from an assay of pooled screening of cells from different genetic backgrounds. In some embodiments, the first plurality of images is a plurality of barcoding images, while the second plurality of images is a plurality of marker-based/marker-free readout image. In some embodiments, the first plurality of images is a plurality of marker-based/marker-free readout images, while the second plurality of images is a plurality of barcoding images. In some embodiments, multiple rounds or acquisitions of marker-based/marker-free readout images may be taken using different imaging/staining paradigms, while the last plurality of images is a plurality of barcoding images.

At block 602, an exemplary system (e.g., one or more electronic devices) generates a first reference coordinate space of the first plurality of images. In other words, the system assigns coordinate values of the first reference coordinate space to pixels of at least one of the first plurality of images. The first plurality of images can comprise one or more sets of images corresponding to one or more acquisitions. The reference coordinate space can be computed at the well level. Specific locations of the well can be assigned values on the first reference coordinate space. As an example, the center of the well can be assigned as the origin (i.e., 0) of the first reference coordinate space. Further, the left- and right-most points of the well can be assigned the values of −X and X on the x-axis and the top- and bottom-most points of the well can be assigned the values of −Y and Y on the y-axis, where X and Y are predefined numbers. In some embodiments, the values of X and Y are based on physical dimensions of the field of view. In some embodiments, the values of X and Y are obtained from image acquisition metadata, which can be provided by the imager.

In some embodiments, the values of coordinates in pixel space can be X=image_size_x (in pixels) and Y=image_size_y (in pixels). In pixel space, the coordinates are with respect to the image dimensions (in pixels). In physical dimensions (i.e., the first coordinate space), the system constructs a coordinate space that is with respect to the microscope stage. Based on the physical dimensions (e.g., field dimensions in micrometers) of the field from the microscope (e.g., metadata), the system can translate the pixel space to the physical space obtained from the microscope

Each image of the first plurality of images can be associated with the first reference coordinate space. In some embodiments, the system detects one or more physical characteristics of the well in an image (e.g., edge of the well, shape of the well, location of the well), and associates the image with the first reference coordinate space accordingly. With reference to FIG. 8, the system can detect the leftmost point and rightmost point of the well in the images, and assign the values of −X and X accordingly. Further, the system can compute the overlap ratio (e.g., between the images 802 and 808). The overlap ratio can be part of the metadata information from the imager (e.g., the microscope metadata) or can be calculated using redundant image information between the two images. Based on the overlap ratios and the specific locations on the well, the system can assign coordinate values (e.g., X, Y) to pixels of the images.

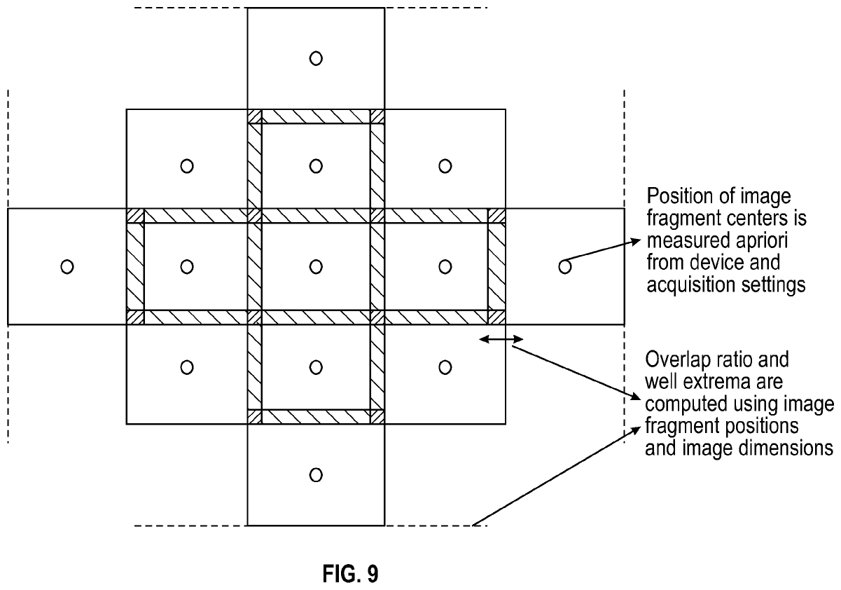

In some embodiments, the system can compute a global coordinate space with respect to the well position in the microscope stage in physical dimensions (e.g., using metadata of the imager). For example, with reference to FIG. 9, the position of the center of each field-of-view image can be measured a priori based on the device and acquisition settings, and the overlap ratio and well extrema are computed using the positions of the field-of-view images and the known image dimensions. In some embodiments, the first coordinate space can be artificially simulated using the pixel's physical dimensions and overlap ratios. For example, in cases where the microscope device metadata does not contain the information for positions (i.e. physical coordinate locations) of the field of view images, the coordinate space can be created by identifying the order of field image acquisition in the well, the image sizes, and the overlap ratio.

At 604, the system extracts a first patch of the first plurality of images. In some embodiments, block 604 includes blocks 606 and 608, as described below.

At block 606, the system selects one or more images that capture one or more landmarks (i.e., marker images) from the first plurality of images. The landmarks can be information within the wells such as one or more nuclei, one or more cells, one or more beads (obtained from fluorescence markers or segmentation from bright-field or quantitative phase contrast images). The landmarks can also be information of the well such as the boundary of the well and the center of the well. In some embodiments, the selection of marker images can be performed using one or more machine-learning models. With reference to FIG. 8, the marker images may include image 802 (which captures a well boundary), 806 (which captures the center of the well), 816 (which captures objects or landmarks within the well).

At block 608, the system extracts the first patch from the one or more marker images. The first patch may be one of the marker images, or a portion of one of the marker images. The patch can be extracted such that it captures a particular object or marker or a particular location of the well (e.g., center of the well). The size of the patch is determined such as whether the patch is large enough to capture the maximum allowable tolerance limit of motion/shift between the acquisitions, as described below with reference to block 612. In some embodiments, the size and location of the patch are determined empirically based on an allowable tolerance threshold. For example, if the system aims to allow up to a shift of 1 mm between the two acquisitions, the system can take a patch size corresponding to x*1 mm in the corresponding image space where x>1. The location of the patch can be anywhere provided we have the patch size corresponding to the tolerance value. In some embodiments, the system extracts the first patch by sparse sampling of the marker images either at random locations in the well or at a fixed location between the two acquisitions. For example, a well can be covered by hundreds of images. Sparse sampling involves choosing a subset of these images corresponding to random physical locations in a well to obtain the first patch. The patch could also be constructed by combining multiple images corresponding to a fixed well location (e.g., the center of the well)

At block 610, the system generates a second reference coordinate space of the second plurality of images. In other words, the system assigns coordinate values of the second reference coordinate space to pixels of at least one of the second plurality of images. The second plurality of images is of the same well on the culture plate. As discussed above, the second plurality of images can be from a second image acquisition. The first and second acquisitions may be different in terms of coverage of the well, image resolution, image size, and imager type, and the relative position of the well may have been shifted/rotated, but the underlying object(s) being imaged are the same. The generation of the second reference coordinate space at block 610 can be performed in a similar manner as, but independently from, block 602.

At block 612, the system extracts a second patch of the second plurality of images. The extraction of the second patch can be performed in a similar manner as block 604. For example, one or more marker images can be selected from the second plurality of images. The second patch can then be extracted from the one or more marker images. The second patch can be extracted such that it captures the same object or marker or the same location of the well (e.g., center of the well) captured in the first patch.

The size of the first and second patches are determined such as whether the patches are large enough to capture the maximum allowable tolerance limit of motion/shift between the acquisitions. For example, if both the first patch and the second patch capture the center of the well, both patches need to be large enough to capture translation, rotation and scaling around the center of the well in both the acquisitions.

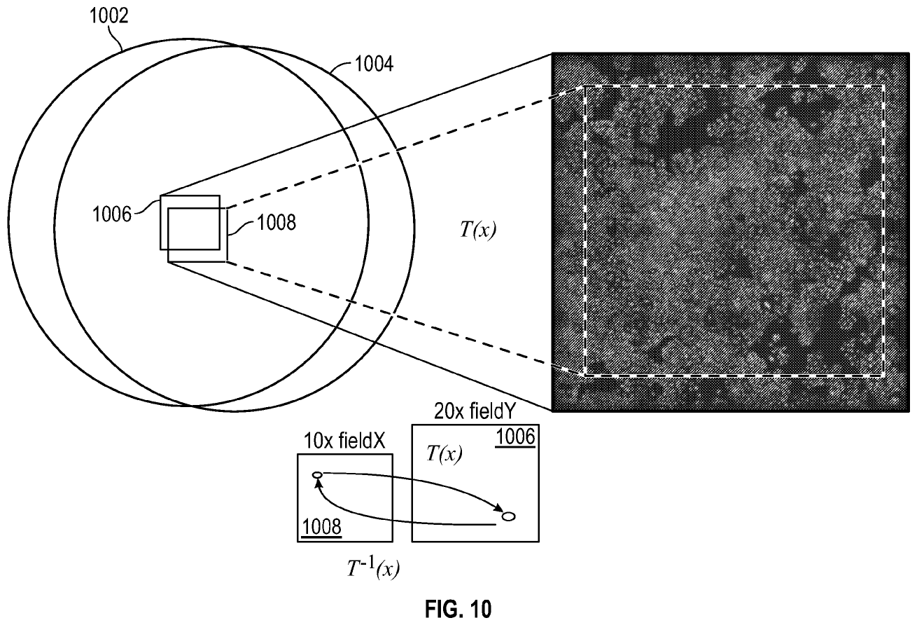

FIG. 10 illustrates two exemplary patches from two different image acquisitions, in accordance with some embodiments. The circle 1002 represents the position of a well during the first image acquisition, and the circle 1004 represents the position of the same well during the second image acquisition. As shown, the well has shifted (e.g., due to movement of the plate or the locations of the imagers) between the two acquisitions. The patch 1006 is selected from the first image acquisition, in which barcoding images are taken. The patch 1008 is selected from the second image acquisition, in which marker-based/marker-free readout images are taken. The two patches also have different resolutions and coverage of the well. In the depicted example, the two patches are selected such that they include the same object. As shown in FIG. 10, the dot in 1008 and the dot in 1006 correspond to the same physical point or location. Affine functions T(x) and T⁻¹(x) can be computed to translate between the coordinate spaces of the two acquisitions, as described below.

At 614, the system computes an affine transformation function between the first patch and the second patch to obtain a plurality of transformation parameters. The transformation parameters can comprise one or more of: a translation parameter, a scaling parameter, and a rotation parameter.

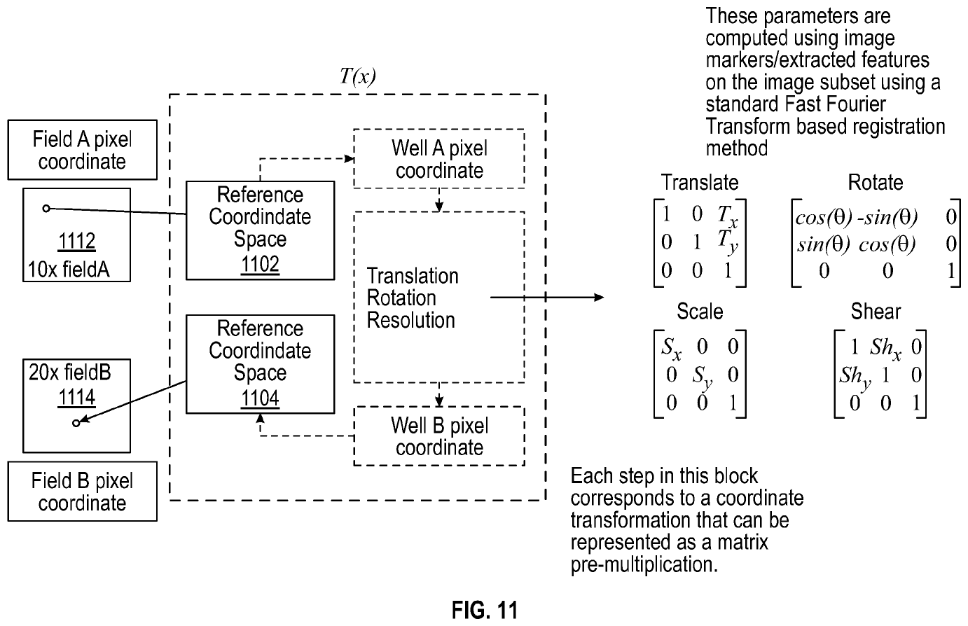

FIG. 11 illustrates an exemplary set of the transformation functions, in accordance with some embodiments. Each of the transformations T₁(x)-T₅(x) can be represented as a matrix multiplication operation where T₁(x)=T₁*x, where T₁ is an affine matrix.

With reference to FIG. 11, a first patch 1112 is from a first image acquisition and is associated with a first reference coordinate space 1102, which can be generated in block 602. T₁(x) refers to the transformation from Field A pixel coordinate (e.g., pixel values) to the reference coordinate space 1102. As discussed above, the values in the first reference coordinate space 1102 can be based on physical dimensions (e.g., physical dimensions of the field of view).

In some embodiments, an additional transformation T₂(x) is performed. T₂(x) refers to the transformation from the first reference coordinate space 1102 to the Well A pixel coordinate. The first reference coordinate space 1102 denotes the coordinate space with respect to the microscope stage as origin. Well pixel coordinate denotes the coordinate space with the top-left corner (could be bottom-left as well) of the well image (in pixels) as origin. The transformation T₂(x) between stage and well pixel coordinates is typically a scale+translation transform that is computed based on the dimensions of the microscope stage and the dimensions of the well image.

In FIG. 11, a second patch 1114 is from a second image acquisition and is associated with a second reference coordinate space 1104, which can be generated in block 610. The pixel in patch 1112 and the pixel in patch 1114 correspond to the same physical point (e.g., same point on the well, same object in the well) but are associated with two different reference coordinate spaces. T₅(x) refers to the transformation from the second reference coordinate space 1104 to the Field B pixel coordinate. In other words, in block 606, T₅⁻¹(x) is obtained in block 610 to transform an image from the second image acquisition to the second reference coordinate space 1104. As discussed above, the values in the second reference coordinate space 1104 can be based on physical dimensions (e.g., physical dimensions of the field of view). Further, T₄(x) refers to the transformation from Well B pixel coordinate to the reference coordinate space 1104, while T₄⁻¹(x) refers to the transformation from the second reference coordinate space 1104 to the Well B pixel coordinate.

At block 614, T₃(x) is calculated based on the transformed patch 1112 (transformed with T₁ and T₂) and the transformed patch 1114 (transformed with T₅⁻¹ and T₄⁻¹). T₃(x) refers to the transformation from Well A pixel coordinate to Well B pixel coordinate, computed from patch information using Fast Fourier transform based registration method. The transformation parameters comprises a translate matrix, a rotate matrix, a scale matrix, and a shear matrix in the depicted example. These parameters are computed using common landmarks (e.g., a specific location on the well, a cell) in the patches using a standard Fast Fourier Transform based registration method.

In other words, T₃(x) is generated based on the two patches to transform between the pixel coordinates of the two reference coordinate spaces. Each arrow between blocks in FIG. 11 involves a coordinate transformation operation. For example, given a field A pixel coordinate, obtaining the reference space A coordinate involves a transformation operation T₁(x).

In some embodiments, the transformation T₃ in FIG. 11 can be computed by applying the four transformation matrices in order which can be represented as matrix pre-multiplication operation on a coordinate vector x.

$$T 3 ( x ) = T 3 \_translate ( T 3 \_shear ( T 3 \_scale ( T 3 \_rotate ( x ) ) ) ) = T 3 \_translate * T 3 \_shear * T 3 \_scale * T 3 \_rotate * x where each of the T 3 \_ <> are the corresponding matrices shown in FIG . 11. = T 3 * x where T 3 = T 3 \_translate * T 3 \_shear * T 3 \_scale * T 3 \_rotate$$

At 616, the system generates a coordinate transformation function between the first reference coordinate space and the second reference coordinate space based on the plurality of transformation parameters. In the depicted example of FIG. 11, the overall transformation matrix is computed as T=T5*T4*T3*T2*T1. Hence, the end-to-end transformation function T(x) can be represented as a series of matrix pre-multiplications where

$$T ( x ) = {T}_{5} ( {T}_{4} ( {T}_{3} ( {T}_{2} ( {T}_{1} ( x ) ) ) ) ) = {T}_{5} * {T}_{4} * {T}_{3} * {T}_{2} * {T}_{1} * x = T * x$$

$$where T = {T}_{5} * {T}_{4} * {T}_{3} * {T}_{2} * {T}_{1}$$

The images may be further processed to obtain cell representations, such as assigning cellular genetic backgrounds, treatments, etc. to single cells in an image. Any model for feature extraction may be used to obtain cell representations in order to classify between genetic backgrounds of cells in an image. In some embodiments, the system deploys self-supervised learning (SSL), semi-supervised, or unsupervised learning methods. In some embodiments, the system deploys SSL techniques in which the machine-learning model(s) learn from unlabeled sample data, as described in detail herein. For example, the image (e.g., segmented morphological image) may be inputted into a trained self-supervised learning model, which is configured to receive an image and output an embedding (i.e., a vector) representing the image in a latent space. The embedding can be a vector representation of the input image in the latent space. Translating an input image into an embedding can significantly reduce the size and dimension of the original data. The lower-dimension embeddings can be used for downstream processing, as described herein. By obtaining embeddings from the images, the self-supervised model can generate a space-time topological space where directionality is available. For example, each image is transformed into an embedding, which can be mapped into a location in the topological space and time-stamped with the time the image was captured. Accordingly, directionality over space and/or time can be obtained across multiple embeddings in the topological space.

In some embodiments, the self-supervised learning model is a DINO Vision Transformer, a SimCLR model, or any other model that learns from unlabeled sample data. In some embodiments, the unsupervised machine-learning model is a trained contrastive learning algorithm. Contrastive learning can refer to a machine learning technique used to learn the general features of a dataset without labels by teaching the model which data points are similar or different. Contrastive learning models can extract embeddings from imaging data that are linearly predictive of labels that might otherwise be assigned to such data. A suitable contrastive learning model is trained by minimizing a contrastive loss, which maximizes the similarity between embeddings from different augmentations of the same sample image and minimizes the similarity between embeddings of different sample images. For example, the model can extract embeddings from images that are invariant to rotation, flipping, cropping and color jittering.

In some embodiments, the system deploys a classifier to analyze the image(s). In some embodiments, the classifier is a convolutional neural network (CNN) comprising a plurality of layers, such as a DenseNet classifier. For example, the image (e.g., segmented morphological image) may be inputted into the classifier, which is configured to receive an image and output a classification result. In some embodiments, a segmented morphological image of a plurality of cells is inputted into the classifier. In some embodiments, a genetic background label (e.g., a label identifying the genotype of an individual cell of a plurality of cells) is the output of the classifier. In some embodiments, the classification result may comprise associating a single cell with a genetic background. An embedding (i.e. vectors) representing the image in a latent space may be obtained from the classifier. In some embodiments, the embedding is obtained from a layer of the plurality of layers of the classifier, prior to the last layer of the classifier. Exemplary DenseNet model architectures are described in Huang et al. (2016), “Densely Connected Convolutional Networks,” arXiv: 1608.06993, the contents of which is incorporated by reference in its entirety. In some embodiments, the plurality of layers are modified to receive a multi-channel fluorescence image as an input, and output a genetic background label. The embedding can be a vector representation of the input image in the latent space. Translating an input image into an embedding using the classifier can significantly reduce the size and dimension of the original data. The embedding or a portion thereof can be used, for example, to evaluate treatment response, such as treatment response of single cells of different genetic backgrounds. In some embodiments, the obtained embedding is further reduced in dimension using the UMAP algorithm to obtain UMAP plots for visualization of the embedding, which can be used for downstream processing, as described herein. The UMAP plot or a portion thereof can be used, for example, to evaluate treatment response, such as treatment response of single cells of different genetic backgrounds.

By obtaining embeddings from the images, the system can generate a space-time topological space where directionality is available. For example, each image is transformed into an embedding, which can be mapped into a location in the topological space and time-stamped with the time the image was captured. Accordingly, directionality over space and/or time can be obtained across multiple embeddings in the topological space. The embedding and subsequent UMAP plot may cluster data points representing single cells, such as single cells of different genetic backgrounds optionally subjected to different treatments (e.g., genetic treatments or chemical treatments), into a particular portion of the embedding and/or UMAP plot. By overlaying images of individual cells over their embedding coordinates, the cells cluster into particular locations in the topological space, and it becomes more clear which features of the cell images (e.g., phenotypes) are causing separation within the model. In some embodiments, the embeddings and/or UMAP plot matches physiological phenotypes with genotypes (e.g., the genetic background) obtained from the classifier as a classification result.

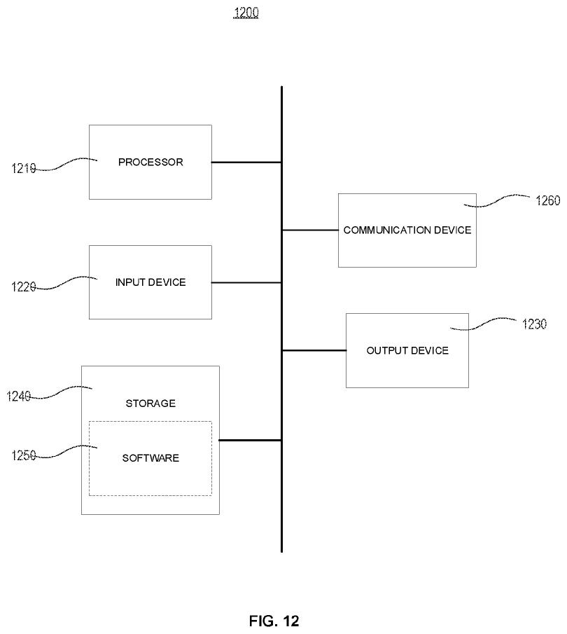

The operations described above with reference to FIG. 6 are optionally implemented by components depicted in FIG. 12. FIG. 12 illustrates an example of a computing device in accordance with one embodiment. Device 1200 can be a host computer connected to a network. Device 1200 can be a client computer or a server. As shown in FIG. 12, device 1200 can be any suitable type of microprocessor-based device, such as a personal computer, workstation, server or handheld computing device (portable electronic device) such as a phone or tablet. The device can include, for example, one or more of processor 1210, input device 1220, output device 1230, storage 1240, and communication device 1260. Input device 1220 and output device 1230 can generally correspond to those described above, and can either be connectable or integrated with the computer.

Input device 1220 can be any suitable device that provides input, such as a touch screen, keyboard or keypad, mouse, or voice-recognition device. Output device 1230 can be any suitable device that provides output, such as a touch screen, haptics device, or speaker.

Storage 1240 can be any suitable device that provides storage, such as an electrical, magnetic or optical memory including a RAM, cache, hard drive, or removable storage disk. Communication device 1260 can include any suitable device capable of transmitting and receiving signals over a network, such as a network interface chip or device. The components of the computer can be connected in any suitable manner, such as via a physical bus or wirelessly.

Software 1250, which can be stored in storage 1240 and executed by processor 1210, can include, for example, the programming that embodies the functionality of the present disclosure (e.g., as embodied in the devices as described above).

Software 1250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1240, that can contain or store programming for use by or in connection with an instruction execution system, apparatus, or device.

Software 1250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 1200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1200 can implement any operating system suitable for operating on the network. Software 1250 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: Modalities Using the Method of Pooled Screening of Cells from Different Genetic Backgrounds

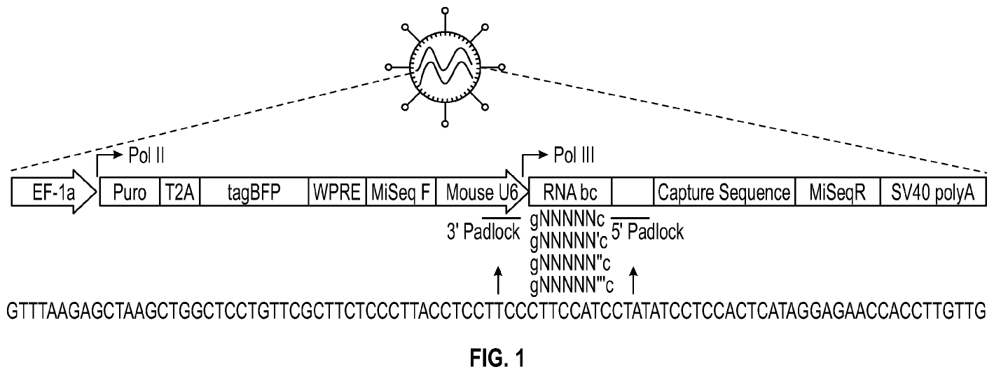

The methods of pooled screening of cells from different genetic backgrounds described in the present application can be used in a variety of different assay modalities. The methods of pooled screening of cells from different genetic backgrounds described herein are also referred to as Visual Village in a Dish (ViViD). In each modality, a vector encoding the unique nucleic acid barcode sequence is introduced into the cells to be assayed. An exemplary vector encoding the unique nucleic acid barcode sequence is shown in FIG. 1.

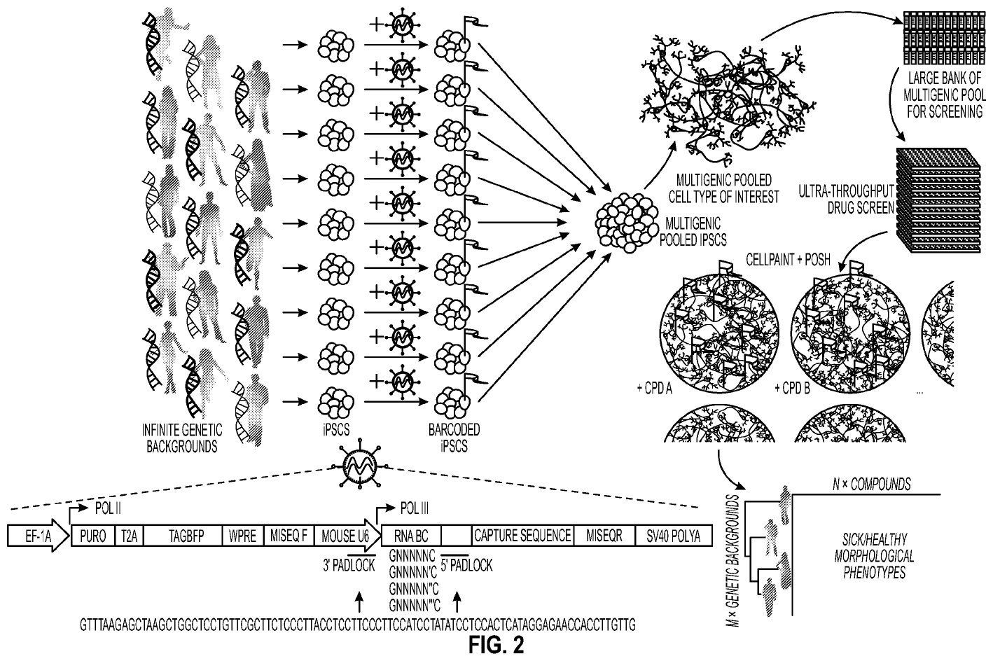

A first modality using the method of pooled screening of cells from different genetic backgrounds described in the present application is shown in FIG. 2. In this modality, a large collection of cell lines is gathered including various genetic backgrounds. These individual lines are then labeled with a unique nucleic acid barcode sequence, which is delivered and integrated into the genome. Cells, such as iPSCs, are then pooled and carried through any assay. In some embodiments, the modality includes performing an ultra-throughput drug screen. At the end of the assay, the barcodes are read, and measured phenotypes are then connected with the genomic DNA that matches the barcode. Lastly, machine learning or other statistical genetics methods are applied to the phenotype/genotype dataset to uncover underlying biology, disease phenotype, drug response, etc.

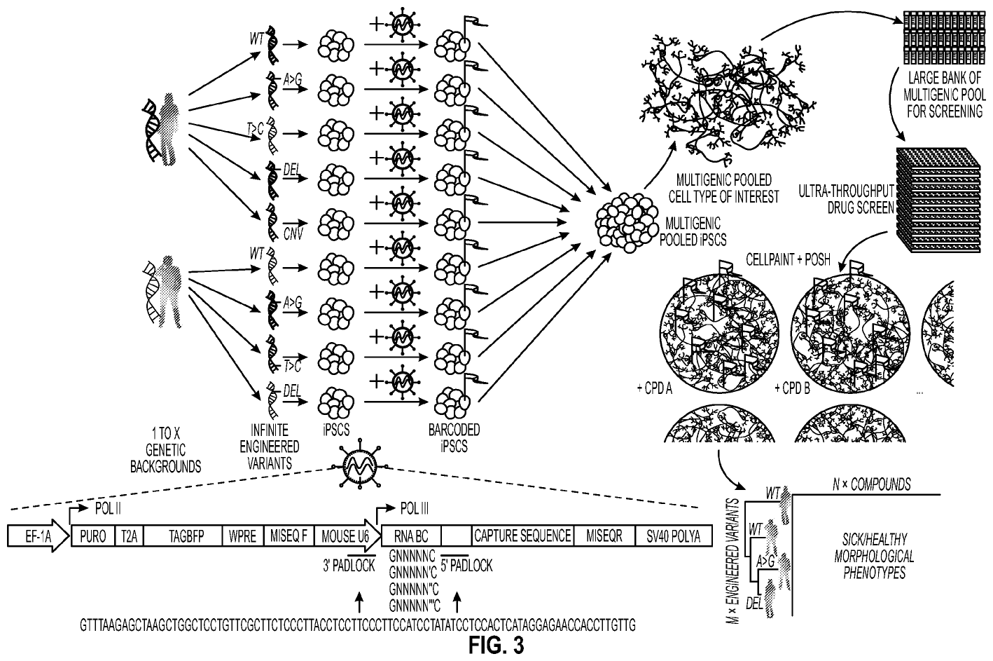

A second modality using the method of pooled screening of cells from different genetic backgrounds described in the present application is shown in FIG. 3. In this modality, rather than pooling several genetic backgrounds as in the first modality, multiple engineered variants from one or more healthy/patient lines are individually labeled. This allows for specifically engineered variants, including purified subclones, to be implemented in the assay. The variant lines, once barcoded, are pooled and carried through any assay as previously described. In some embodiments, the modality includes performing an ultra-throughput drug screen.

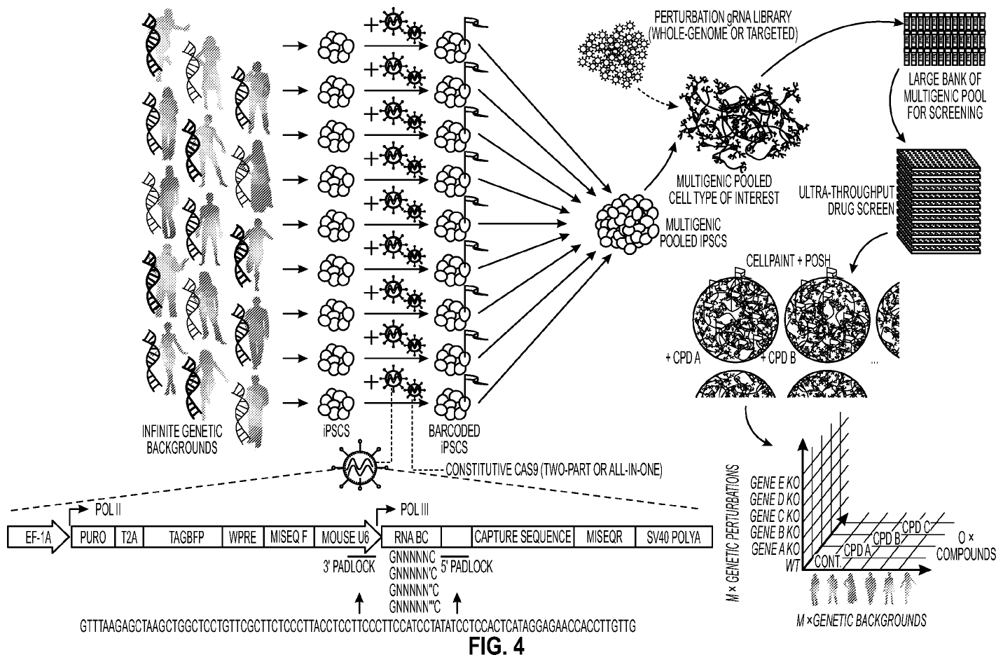

A third modality using the method of pooled screening of cells from different genetic backgrounds described in the present application is shown in FIG. 4. In this modality, standard ViViD is merged with perturbation. Multiple cell lines from several genetic backgrounds are collected and individually labeled with unique nucleic acid barcode sequences. Those cells are also engineered to include a genetic modifying agent, and then treated with a pooled lentiviral library containing gRNAs targeting a selection of, or all, genes. Alternatively, the lentiviral library could be an ‘all-in-one’ vector. The pooled cells are then carried through any assay. In some embodiments, the modality includes performing an ultra-throughput drug screen. Lastly, the combination of genotype and perturbation can be detected and tied back to any cellular phenotype observed.

### Example 2: Analysis of Data Obtained from Pooled Screening of Cells from Different Genetic Backgrounds

Described herein is an analysis pipeline that can be used to analyze the data obtained from the method of pooled screening of cells from different genetic backgrounds described in the present application. The methods of pooled screening of cells from different genetic backgrounds described herein are also referred to as Visual Village in a Dish (ViViD). The analysis pipeline includes nonparametric and parametric learnable methods for detecting and demultiplexing cell barcodes, segmenting cells, aligning image coordinates between acquisitions and other downstream tasks for learning meaningful representations and discovering unknown biology from the data generated from the methods of pooled screening of cells from different genetic backgrounds described herein.

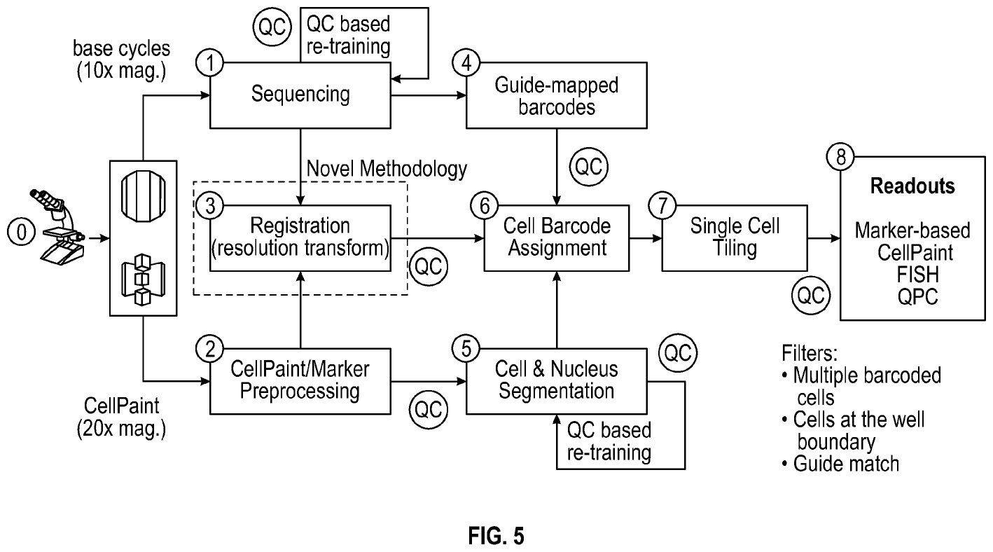

FIG. 5 is a schematic showing the analysis pipeline used to analyze the data obtained from the method of pooled screening of cells from different genetic backgrounds described in the present application. The analysis pipeline involves different sub-components starting from microscopic image acquisition and ending with single-cell image based readouts for downstream analysis. The analysis pipeline involves the following steps:
(0) Barcoding images (sequencing by synthesis) and marker-based/marker-free readout images (CellPaint/antibody stain/FISH/QPC) imaged using a microscope
(1) Detecting and sequencing barcodes from individual base cycles imaged by sequencing-by-synthesis
(2) Preprocessing marker-based/marker-free cellular images for downstream readouts
(3) Computing a transformation between sequencing and marker-based/marker-free readouts for demultiplexing single-cell image based readouts
(4) Mapping the sequenced barcodes to a dictionary of barcodes used in the experiment
(5) Segmentation of nuclei and cell boundaries for identifying single cells in marker-based/marker-free readout images
(6) Assignment of sequenced barcodes to the corresponding single-cells identified by cell-segmentation using transform computed from (3)
(7) Tiling (and masking by cell-segmentations) marker-based/marker-free readout images for downstream single-cell image analysis
(8) Non-exhaustive list of possible single-cell readouts enabled by this pipeline (antibody/fluorescence protein markers, CellPaint, Quantitative phase images, fluorescent in-situ hybridization)

### Example 3: Pooled Screening of Cells from Different Genetic Backgrounds Using a Visual Village in a Dish (ViViD)

This example shows pooled screening of cells from different genetic backgrounds and the analysis thereof using the methods described herein. Specifically, this example shows detecting and demultiplexing of cell barcodes, segmenting cells, and aligning image coordinates between acquisitions from the data generated from the methods of pooled screening of cells from different genetic backgrounds described herein.

### Sample Preparation, Screening, and Cell Culture

Wildtype GM25256 induced pluripotent stem cells (iPSCs) (California Institute for Regenerative Medicine (CIRM)) were expanded using standard Essential 8 (E8), vitronectin, and ReLeSR culture methods. Cells were engineered to contain a doxycycline-inducible neurogenin-2 (doxNGN2) gene in the AAVS1 safe harbor locus the doxNGN2 vector included a connected geneticin-resistance gene, allowing for continuous culture under antibiotic selection of these vectors.

Initial screening was done using ribonucleoprotein (RNP) transfection of guide RNA(gRNA)-bound Cas9, to identify effective gRNAs for the knockout of TSC2 (“TSC2 ko”) and SETD1A genes (“SETD1A ko”), respectively. iPSCs were then subcloned through single or double rounds to achieve pure homozygous or heterozygous knockouts of the target genes. Wt control subclones were taken through the same process, but were derived from the cells that had not received edits during the RNP transfection stage. Homozygous knockouts of SETD1A did not survive in culture, due to the essentiality of at least one copy of the gene for cell survival. Therefore, heterozygous knockouts of SETD1A (“SETD1A het”) were generated using number-designated SETD1A gRNA 3 and SETD1A gRNA4, resulting in cell lines labeled as heterozygous knockouts of SETD1AG3 (“SETD1AG3 het”), and heterozygous knockouts of SETD1AG4 (“SETD1AG4 het”), respectively. Heterozygous knockouts of TSC2 (“TSC2 het”).

Lentiviral vectors containing non-targeting gRNAs were synthesized. The multiple engineered lines described above were each labeled with a unique gRNA label, which was recorded as the genotype's specific “barcode”. Cells that had lentiviral integration of the gRNA-expression transcript were selected with puromycin. The lentiviral vector also contained a feature extraction component, allowing the unique labels to be read during single cell RNAseq (scRNAseq) analysis.

After each subclone was uniquely labeled, the multiple engineered lines were pooled together for all downstream assays. Cells were then differentiated into neurons using induction of the NGN2 gene. Briefly, cells were dissociated via accutase and plated at Day −2 in E8 medium+Y27632, then fed on Day −1 with only E8. On Day 0, cells were treated to induce NGN2 expression on Day 1 and Day 2.

On Day 2, culture plates were treated with 0.1% polyethylenimine (PEI) in cell culture water and incubated at 37° C. overnight. On Day 3, PEI plates were thoroughly aspirated and left to dry. The PEI plates were then coated with laminin in PBS for 1 hour at 37° C. Neural progenitors were then dissociated for 5 minutes at 37° C. with accutase, and resuspended in NGN2 medium. Cells were filtered and plated out at a varying density.

For the neuron-only cultures (e.g., scRNAseq samples), laminin coating solution was aspirated, and neural progenitor cells were plated on the PEI/laminin coated culture plates in NGN2 medium.

For the neuron-astrocyte co-cultures (e.g., imaging samples), neural progenitor cells were plated simultaneously with rat astrocytes (Lonza) onto the PEI/laminin coated culture plates. Each well received neurons and rat astrocytes at a ratio of 3:2, all in NGN2 medium. Medium was changed on a Monday/Wednesday/Friday schedule, with Day 6 starting on Monday. On each feed day, ⅓ of the media was removed from each well and replaced with fresh medium. Starting on Day 11, cells were treated with NGN2 medium without doxycycline, continuing the Monday/Wednesday/Friday schedule.

### Imaging and Segmentation

Starting on Day 20, cells were treated with either DMSO control or 100 nM rapamycin. Rapamycin treatments were replenished on Day 22 and Day 24, for a final paraformaldehyde (PFA) fixing of plates on Day 27 (total treatment duration of 7 days). The fixed cells were washed, and the respective background genome labels (“barcodes”) were reverse transcribed and amplified by rolling circle amplification with gap filling.

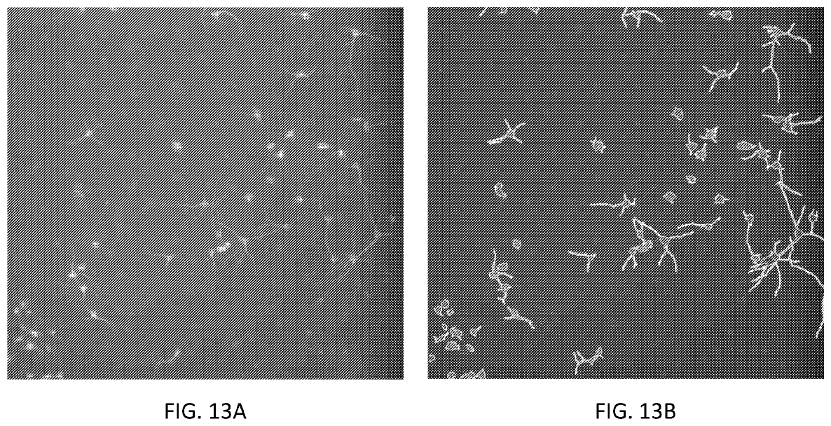

Cells were stained with Hoescht (1:200,000), 1:1,000 rab-phS6 antibody (Cell Signaling Tech), and 1:2,500 ch-MAP2 antibody (Novus) overnight at 4° C., corresponding secondary antibodies, and imaged via Nikon Ti2 with lumencor celesta excitation. FIG. 13A shows an exemplary image of the stained cells in a co-culture of neurons and astrocytes.

The cell images were then segmented by nuclei and cell boundaries, as shown in FIG. 13B, for identifying neuronal single cells in a readout image. Briefly, nuclei segmentation masks were generated by thresholding the DAPI channel followed by applying a watershed transform to obtain labeled nuclei instances. The neuronal cells were then segmented by thresholding MAP2 channel followed by a series of morphological operations and applying the watershed transform using nuclei segmentation masks as seeds.

### Detecting and Sequencing Barcodes

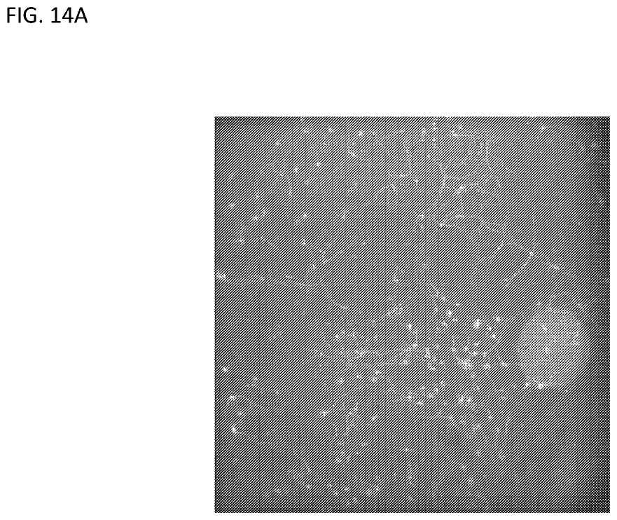

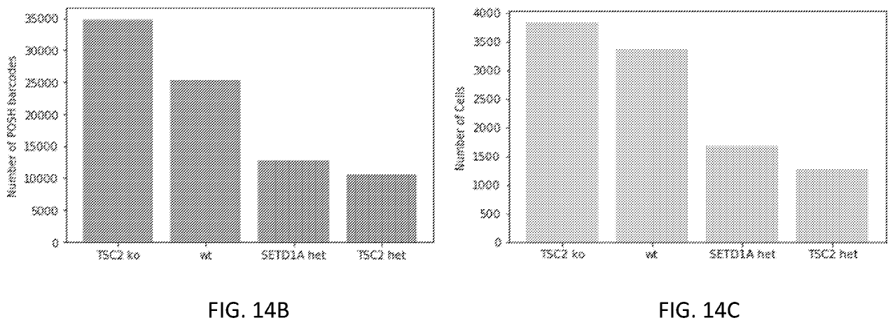

After imaging, cells underwent several cycles of in situ sequencing-by-synthesis until entire gRNA barcode labels, corresponding to the cell genetic backgrounds, were measured in the respective cells. An exemplary image of the in situ sequencing is shown in FIG. 14A. The sequenced barcodes were mapped to a dictionary of barcodes used in the experiment. The barcode sequences were computed from the sequencing-by-synthesis images using a peak detection algorithm followed by intensity based classification of nucleotide bases based on the peak fluorescence channel. FIG. 14B shows the number of POSH (e.g., pooled optical screening) barcodes totalled for each cell line genetic background, and the corresponding number of cells, based on the number of POSH barcodes is shown in FIG. 14C.

### Transformation

Next, a transformation between sequencing and marker-based/marker-free readouts for demultiplexing single-cell image based readouts was computed. The sequenced barcodes were assigned to the corresponding single-cells identified by cell-segmentation using the computed transformation. Briefly, the well images from the first cycle of sequencing-by-synthesis imaging run and the phenotype (MAP2+DAPI) imaging run were then registered to compute a transformation function. The barcode locations in the first cycle of sequencing-by-synthesis are then transformed to the phenotype images using the computed transformation function, and were assigned to single cells based on their segmentation labels. Once the barcodes were assigned to the cells, single neuronal cell images were generated using the segmentation mask and were stored for subsequent modeling. In order to train the supervised classifier, samples from one well were used to create a test dataset, and the rest of the wells were used for training. A supervised DenseNet model was trained using the dataset with a cross entropy loss function to classify between genetic backgrounds given a single cell input. In order to obtain low-dimensional representation of these single cell images, the activation output layer before the last layer of the network was utilized as low-dimensional feature embeddings for visualization. These embeddings were then further reduced in dimension using the UMAP algorithm to obtain two dimensions (e.g., “UMAP1” and “UMAP2”) for visualization.

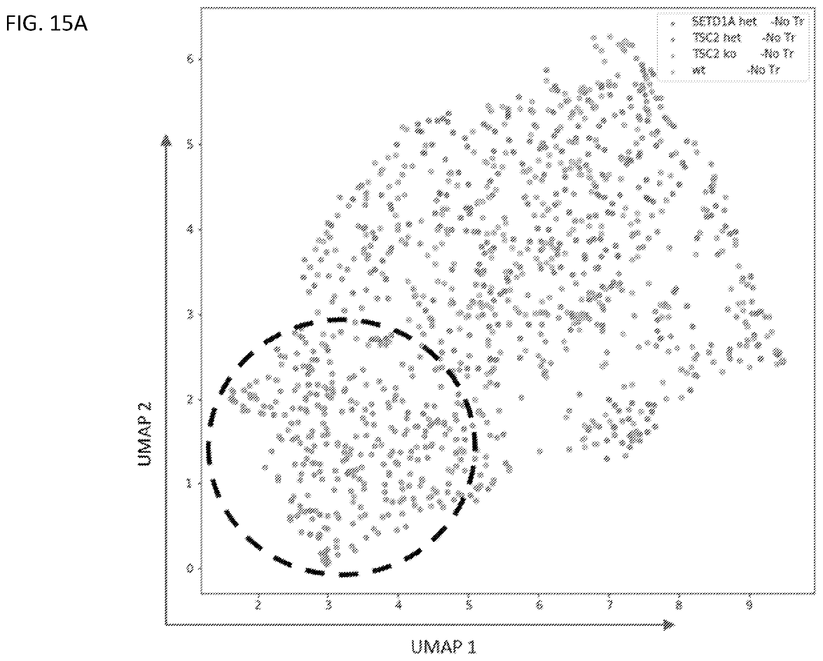

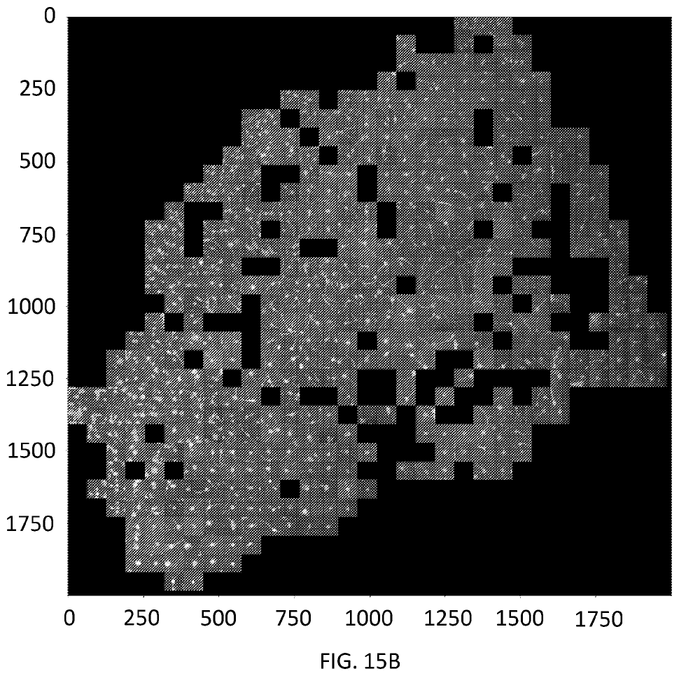

The supervised DenseNet model was able to achieve a 45.5% classification accuracy on the test dataset. FIG. 15A shows an exemplary transformation visualizing the barcode sequencing and imaging data (e.g., embedding, “UMAP1 and UMAP2”) of untreated (“No Tr”) cells. By overlaying representative image tiles of individual cells over their embedding coordinates (FIG. 15B), it becomes more clear which features of the cell images are causing separation within the model. Particularly, lower UMAP1/higher UMAP2 (top left of FIGS. 15A and 15B) regions included more dense regions of the pooled cells, with a mix of multiple genotypes, resulting in cells of multiple genotypes in a single tile. Alternatively, a clear difference in soma size and neurite shape emerges in low UMAP 1/low UMAP 2 (bottom left of FIGS. 15A (circle with dashed lines) and 15B) compared to high UMAP1 (middle right of FIGS. 15A and 15B), matching the physiological phenotypes that neurons exhibit in patient hamartomas. In summary, the UMAP visualizations confirmed that the TSC2 ko cells have a different feature embedding space, indicated by a circle with dashed lines in FIG. 15A, compared to the rest of the cells.

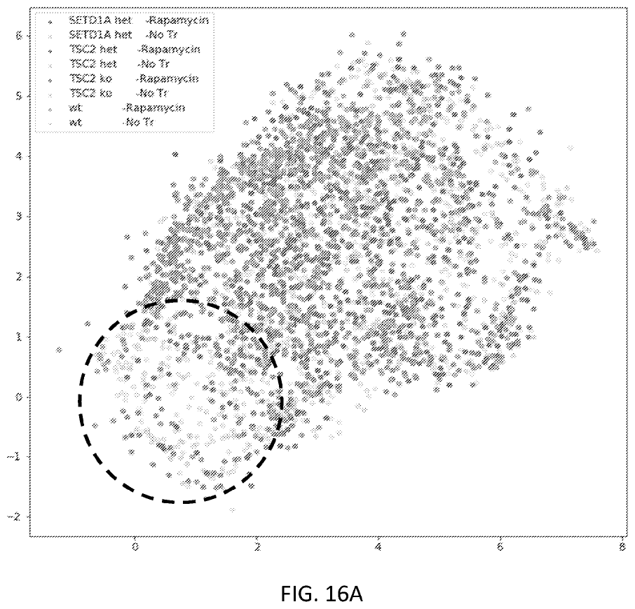

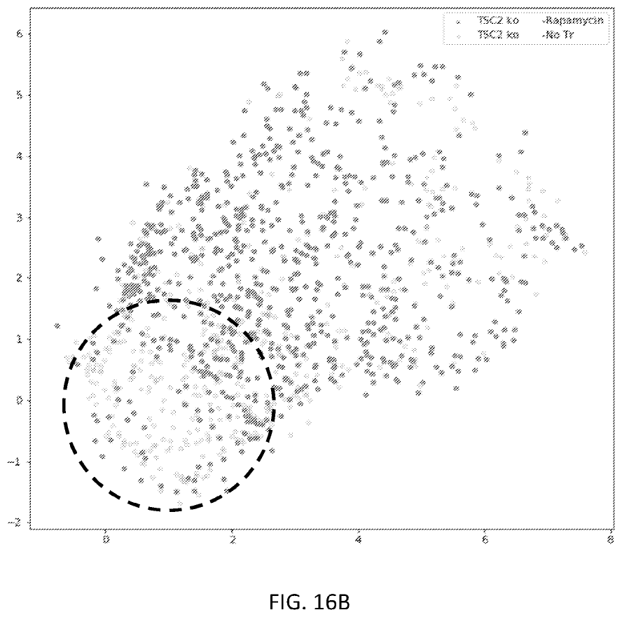

FIGS. 16A-16B show exemplary transformations visualizing the barcode sequencing and imaging data (e.g., embedding) of untreated cells (“No Tr”) and cells treated with rapamycin (“Rapamycin”). The bottom left cluster of FIGS. 16A and 16B (indicated by a circle with dashed lines) is predominantly populated by untreated TSC2 ko cells, while untreated wt cells, SETD1A het cells, and rapamycin-treated TSC2 ko neurons all group to other regions of the embedding.

scRNAseq Analysis

On Day 14, neurons were treated with 6 different treatments: Rapamycin (100 nM), Everolimus (100 nM), Lonafarnib (100 nM), Iadademstat (100 nM), DMSO (1:100,000 dilution to match treatments), and untreated. Feeds were conducted so target dilutions were made after ⅓ medium changes. Cells were treated once again on Day 16 with the same treatments. On Day 17, neurons were dissociated via accutase at 37° C. for 30 minutes. Accutase was deactivated with NGN2 and cells were filtered and counted. Cells were resuspended in media and run through standard 10× scRNAseq processing with PCR-amplified feature extraction option. Sequencing libraries were aligned to Hg38 transcription map using standard CellRanger pipeline (10×). Genotype-labeling non-targeting gRNAs (e.g., “features”) were assigned to cells based on the following: 1) the highest-count barcode was assigned to a given cell if it made up >55% of total barcode reads; 2) if the second-highest-count barcode made up >40% of reads, the cell was labeled as a “multiplet” (taking priority over “1”); and, 3) if no barcode counts were detected, the cell was labeled “unassigned”. By labeling the genetic background and tracking the treatment well that each pool came from, a dataset combining 6 treatments with 14 genetic backgrounds was generated.

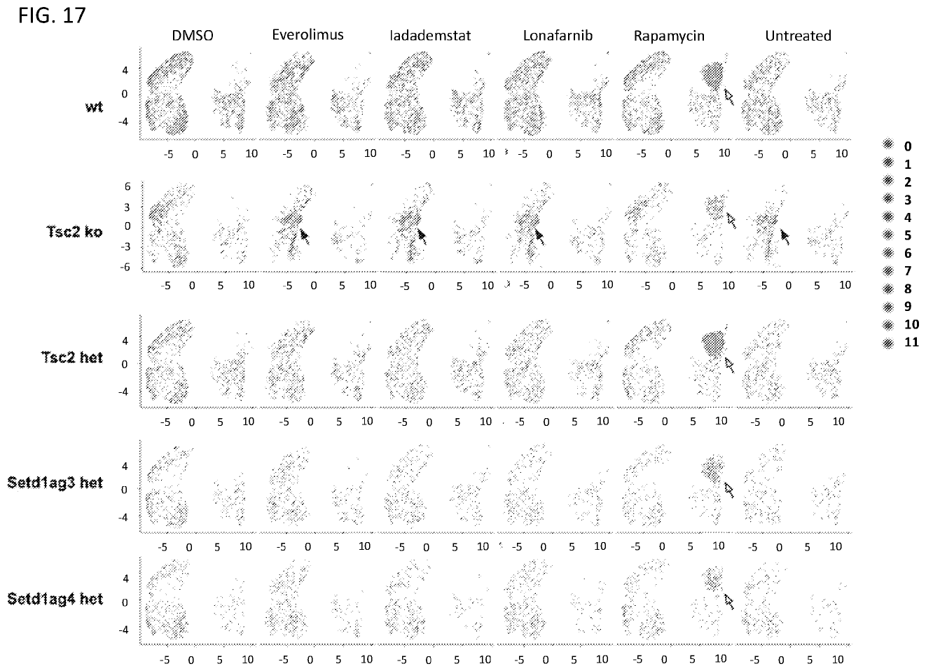

FIG. 17 shows an exemplary embedding of pooled ViViD-labelled cells, with barcodes and transcriptional signatures measured using scRNAseq, of untreated cells and cells treated with Rapamycin, Everolimus, Lonafarnib, Iadademstat, and DMSO. The TSC2 ko neurons are shown in cluster 6 (solid arrows). Rapamycin treatment pushed all cells of all gentoypes, including the TSC2 ko neurons, into a new population (open arrows).

Overall, these data indicate that the Visual Village in a Dish (ViViD) pooled optical screening platform of cells from different genetic backgrounds is capable of discovering novel biology using a large-scale population-genetics based assay on specific cell types without convoluting factors.

### Exemplary Embodiments

The following embodiments are exemplary and are not intended to limit the scope of the invention described herein.

Embodiment 1. A method of pooled screening of cells from different genetic backgrounds, comprising:
a) labeling two or more populations of cells of different genetic backgrounds with two or more unique nucleic acid barcode sequences, each unique nucleic acid barcode sequence corresponding to a different population of cells;
b) combining the two or more populations of cells to obtain a single mixed population of cells;
c) performing in situ single-cell sequencing on the cells; and
d) analyzing known or identifying new phenotypes of the cells in the mixed population.

Embodiment 2. The method of embodiment 1, wherein the two or more populations of cells are from different cell lines.

Embodiment 3. The method of embodiment 2, wherein the different cell lines are healthy cell lines.

Embodiment 4. The method of embodiment 2, wherein the different cell lines are patient cell lines.

Embodiment 5. The method of embodiment 2, wherein the different cell lines are isogenically engineered cell lines.

Embodiment 6. The method of embodiment 2, wherein the different cell lines include any combination of healthy, patient, and isogenically engineered cell lines.

Embodiment 7. The method of any one of embodiments 1-6, wherein the cells are induced pluripotent stem cells (iPSCs).

Embodiment 8. The method of embodiment 7, wherein the iPSCs are differentiated prior to analyzing the phenotype.

Embodiment 9. The method of embodiment 8, wherein the method further comprises culturing the cells prior to analyzing the phenotype.

Embodiment 10. The method of any one of embodiments 1-9, wherein the single mixed population of cells is on a substrate or in three-dimensional culture.

Embodiment 11. The method of embodiment 10, wherein the substrate is a cell culture dish.

Embodiment 12. The method of any one of embodiments 1-11, wherein the method further comprises performing single cell RNAseq.

Embodiment 13. The method of any one of embodiments 1-12, wherein the method further comprises growing the two or more populations of cells for two or more generations prior to step b).

Embodiment 14. The method of any one of embodiments 1-13, wherein the method comprises stably integrating the unique nucleic acid barcode sequences into the genomes of the two or more populations of cells.

Embodiment 15. The method of embodiment 14, wherein the unique nucleic acid barcode sequences are delivered into the cell using a virus.

Embodiment 16. The method of embodiment 15, wherein the virus is a lentivirus.

Embodiment 17. The method of embodiment 15 or embodiment 16, wherein the virus encodes a selectable marker.

Embodiment 18. The method of embodiment 17, wherein the selectable marker is an antibiotic resistance gene.

Embodiment 19. The method of any one of any one of embodiments 15-18, wherein the virus encodes a fluorescent protein.

Embodiment 20. The method of any of embodiments 1-19, wherein each unique nucleic acid barcode sequence is at least 1 base pair in length.

Embodiment 21. The method of embodiment 20, wherein each unique nucleic acid barcode sequence is 1 to about 18 base pairs in length.

Embodiment 22. The method of embodiment 20, wherein each unique nucleic acid barcode sequence is 8 base pairs in length.

Embodiment 23. The method of any one of embodiments 1-22, wherein the two or more populations of cells are sequenced prior to labeling with the unique nucleic acid barcode sequences.

Embodiment 24. The method of embodiment 23, wherein the sequencing is whole genome sequencing.

Embodiment 25. The method of any one of embodiments 1-24, wherein the two or more populations of cells were obtained from related individuals.

Embodiment 26. The method of embodiment 25, wherein the two or more populations of cells were obtained from humans.

Embodiment 27. The method of any one of embodiments 1-26, comprising labeling ten or more populations of cells of different genetic backgrounds with ten or more unique nucleic acid barcode sequences, each unique nucleic acid barcode sequence corresponding to a different population of cells.

Embodiment 28. The method of any one of embodiments 1-27, wherein analyzing the phenotype of the cells comprises an assay selected from the group consisting of high content imaging, calcium imaging, immunohistochemistry, cell morphology imaging, protein aggregation imaging, cell-cell interaction imaging, live cell imaging, and any other imaging-based assay modality.

Embodiment 29. The method of any one of embodiments 1-28, wherein step d) comprises analyzing the phenotype of the cells by capturing a microscopic image or a time series of microscopic images of the cells and evaluating phenotypic features presented in the image or images.

Embodiment 30. The method of embodiment 29, wherein the method further comprises a computer-implemented technique for aligning between a first plurality of images and a second plurality of images, comprising:
generating a first reference coordinate space of the first plurality of images, wherein the first plurality of images is of a well on a culture plate;
extracting a first patch of the first plurality of images;
generating a second reference coordinate space of the second plurality of images, wherein the second plurality of images is of the well on the culture plate;
extracting a second patch of the second plurality of images;
computing an affine transformation function between the first patch and the second patch to obtain a plurality of transformation parameters; and
generating a coordinate transformation function between the first reference coordinate space and the second reference coordinate space based on the plurality of transformation parameters.

Embodiment 31. A computer-implemented method for aligning between a first plurality of images and a second plurality of images, comprising:
generating a first reference coordinate space of the first plurality of images, wherein the first plurality of images is of a well on a culture plate;
extracting a first patch of the first plurality of images;
generating a second reference coordinate space of the second plurality of images, wherein the second plurality of images is of the well on the culture plate;
extracting a second patch of the second plurality of images;
computing an affine transformation function between the first patch and the second patch to obtain a plurality of transformation parameters; and
generating a coordinate transformation function between the first reference coordinate space and the second reference coordinate space based on the plurality of transformation parameters.

Embodiment 32. The method of embodiment 31, wherein the first plurality of images is a plurality of barcoding images.

Embodiment 33. The method of embodiment 31 or embodiment 32, wherein the second plurality of images is a plurality of marker-based/marker-free readout image.

Embodiment 34. The method of any one of embodiments 31-33, wherein the first plurality of images and the second plurality of images provide different coverage of the well.

Embodiment 35. The method of any one of embodiments 33-34, wherein the first plurality of images and the second plurality of images are taken at different times.

Embodiment 36. The method of any one of embodiments 31-35, wherein the first plurality of images and the second plurality of images have different resolutions.

Embodiment 37. The method of any one of embodiments 31-36, wherein the first plurality of images is taken by a first microscope and the second plurality of images is taken by a second microscope.

Embodiment 38. The method of embodiment 37, wherein the first microscope is a fluorescent microscope.

Embodiment 39. The method of embodiment 37, wherein the second microscope is a non-fluorescent microscope.

Embodiment 40. The method of any one of embodiment 31-39, wherein the first plurality of images is captured by a first imager.

Embodiment 41. The method of embodiment 40, further comprising:
detecting one or more physical characteristics of the well in the first plurality of images; and
generating the first reference coordinate space based on the detected one or more physical characteristics.

Embodiment 42. The method of embodiment 41, wherein the one or more physical characteristics of the well comprises: a shape of the well, an edge of the well, a location of the well.

Embodiment 43. The method of any one of embodiments 40-42, wherein two or more images in the first plurality of images are offset from each other by an overlap ratio, and wherein the first reference coordinate space is generated based on the overlap ratio.

Embodiment 44. The method of any one of embodiments 40-43, wherein the first reference coordinate space is generated based on metadata of the first imager.

Embodiment 45. The method of any one of embodiments 40-44, further comprising:
selecting one or more marker images from the first plurality of images based on one or more landmarks captured in the one or more images, wherein the first patch is obtained from the one or more marker images from the first plurality of images.

Embodiment 46. The method of embodiment 45, wherein the one or more landmarks comprise: one or more cells, one or more well boundaries, one or more beads, one or more nuclei, or any combination thereof.

Embodiment 47. The method of any one of embodiment 31-46, wherein the second plurality of images captured by a second imager.

Embodiment 48. The method of embodiment 47, further comprising:
detecting one or more physical characteristics of the well in the second plurality of images; and
generating the second reference coordinate space based on the detected one or more physical characteristics.

Embodiment 49. The method of embodiment 48, wherein the one or more physical characteristics of the well comprises: a shape of the well, an edge of the well, a location of the well.

Embodiment 50. The method of any one of embodiments 47-49, wherein two or more images in the second plurality of images are offset from each other by an overlap ratio, and wherein the second reference coordinate space is generated based on the overlap ratio.

Embodiment 51. The method of any one of embodiments 47-50, wherein the second reference coordinate space is generated based on metadata of the second imager.

Embodiment 52. The method of any one of embodiments 47-51, further comprising: selecting one or more marker images from the second plurality of images based on one or more landmarks captured in the one or more images, wherein the second patch is obtained from the one or more marker images of the second plurality of images.

Embodiment 53. The method of embodiment 52, wherein the one or more landmarks comprise: one or more cells, one or more well boundaries, one or more beads, one or more nuclei, or any combination thereof.

Embodiment 54. The method of any one of embodiments 31-53, wherein the first patch covers a center of the first image.

Embodiment 55. The method of any one of embodiments 31-54, wherein the second patch covers a center of the second image.

Embodiment 56. The method of any one of embodiments 31-55, wherein at least a portion of the first patch and at least a patch of the second patch correspond to the same subject.

Embodiment 57. The method of any one of embodiments 31-56, wherein the transformation parameters comprise one or more of: a translation parameter, a scaling parameter, and a rotation parameter.

Embodiment 58. The method of any one of embodiments 31-57, wherein the first and second images were obtained from an assay of pooled screening of cells from different genetic backgrounds, comprising:
a) labeling two or more populations of cells of different genetic backgrounds with two or more unique nucleic acid barcode sequences, each unique nucleic acid barcode sequence corresponding to a different population of cells;
b) combining the two or more populations of cells to obtain a single mixed population of cells;
c) performing in situ single-cell sequencing on the cells; and
d) analyzing known or identifying new phenotypes of the cells in the mixed population.

Embodiment 59. The method of any one of embodiments 1-58, further comprising utilizing a classifier configured to receive an image and output a classification result.

Embodiment 60. The method of embodiment 59, wherein the classifier comprises a plurality of layers.

Embodiment 61. The method of embodiment 59 or 60, wherein the classifier is a convolutional neural network.

Embodiment 62. The method of any one of embodiments 59-61, wherein the classifier is a DenseNet classifier.

Embodiment 63. The method of any one of embodiments 59-62, wherein the classification result is a classification based on the genetic background of each single cell captured in the image.

Embodiment 64. The method of any one of embodiments 59-63, further comprising generating an embedding of the image.

Embodiment 65. The method of embodiment 64, wherein the embedding is generated from an activation output layer prior to the last layer of the classifier.

Embodiment 66. The method of embodiment 64 or 65, wherein the embedding is reduced in dimension using a linear dimensionality reduction method.

Embodiment 67. The method of any one of embodiments 64-66, wherein the embedding is reduced in dimension using a UMAP (Uniform Manifold Approximation and Projection for Dimension Reduction) algorithm to obtain one or more UMAP plots for visualization of the embedding.

Embodiment 68. The method of any one of embodiments 64-67, further comprising evaluating a treatment based on at least a portion of the embedding.

Embodiment 69. The method of embodiment 67 or 68, further comprising evaluating a treatment based on at least a portion of the one or more UMAP plots.

1-30. (canceled)

31. A method of pooled screening of cells from different genetic backgrounds, comprising:
labeling two or more populations of cells of different genetic backgrounds with two or more unique nucleic acid barcode sequences, each unique nucleic acid barcode sequence corresponding to a different population of cells;
combining the two or more populations of cells to obtain a single mixed population of cells in a well on a culture plate;
performing in situ single-cell sequencing on the cells;
obtaining a first plurality of images, wherein the first plurality of images comprises one or more barcoding images of the well on the culture plate;
obtaining a second plurality of images, wherein the second plurality of images comprises one or more marker-based or marker-free readout images of the well on the culture plate;
generating a first reference coordinate space of the first plurality of images;
extracting a first patch of the first plurality of images;
generating a second reference coordinate space of the second plurality of images;
extracting a second patch of the second plurality of images;
computing an affine transformation function between the first patch and the second patch to obtain a plurality of transformation parameters; and
generating a coordinate transformation function between the first reference coordinate space and the second reference coordinate space based on the plurality of transformation parameters;
identifying cell-specific image data for one or more cells in the single mixed population of cells by correlating at least one image of the first plurality of images with at least one image of the second plurality of images based on the coordinate transformation function; and
identifying one or more phenotypes of the one or more cells based on the cell-specific image data.

32-33. (canceled)

34. The method of claim 31, wherein the first plurality of images and the second plurality of images provide different coverage of the well, are taken at different times, and/or have different resolutions.

35-39. (canceled)

40. The method of claim 31, wherein the first plurality of images is captured by a first imager.

41. The method of claim 40, further comprising:
detecting one or more physical characteristics of the well in the first plurality of images, wherein the one or more physical characteristics of the well comprises: a shape of the well, an edge of the well, a location of the well; and
generating the first reference coordinate space based on the detected one or more physical characteristics.

42. (canceled)

43. The method of claim 40, wherein two or more images in the first plurality of images are offset from each other by an overlap ratio, and wherein the first reference coordinate space is generated based on the overlap ratio.

44. (canceled)

45. The method of claim 40, further comprising:
selecting one or more marker images from the first plurality of images based on one or more landmarks captured in the one or more images, wherein the first patch is obtained from the one or more marker images from the first plurality of images, and wherein the one or more landmarks comprise: one or more cells, one or more well boundaries, one or more beads, one or more nuclei, or any combination thereof.

46. (canceled)

47. The method of claim 31, wherein the second plurality of images captured by a second imager.

48. The method of claim 47, further comprising:
detecting one or more physical characteristics of the well in the second plurality of images, wherein the one or more physical characteristics of the well comprises: a shape of the well, an edge of the well, a location of the well; and
generating the second reference coordinate space based on the detected one or more physical characteristics.

49. (canceled)

50. The method of claim 47, wherein two or more images in the second plurality of images are offset from each other by an overlap ratio, and wherein the second reference coordinate space is generated based on the overlap ratio.

51. (canceled)

52. The method of claim 47, further comprising:
selecting one or more marker images from the second plurality of images based on one or more landmarks captured in the one or more images, wherein the second patch is obtained from the one or more marker images of the second plurality of images, and wherein the one or more landmarks comprise: one or more cells, one or more well boundaries, one or more beads, one or more nuclei, or any combination thereof.

53. (canceled)

54. The method of claim 31, wherein the first patch covers a center of the first image and/or wherein the second patch covers a center of the second image.

55. (canceled)

56. The method of claim 31, wherein at least a portion of the first patch and at least a patch of the second patch correspond to the same subject.

57. The method of claim 31, wherein the transformation parameters comprise one or more of: a translation parameter, a scaling parameter, and a rotation parameter.

58. (canceled)

59. The method of claim 31, further comprising utilizing a classifier configured to receive an image and output a classification result.

60-62. (canceled)

63. The method of claim 59, wherein the classification result is a classification based on the genetic background of each single cell captured in the image.

64. The method of claim 59, further comprising generating an embedding of the image.

65-66. (canceled)

67. The method of claim 64, wherein the embedding is reduced in dimension using a UMAP (Uniform Manifold Approximation and Projection for Dimension Reduction) algorithm to obtain one or more UMAP plots for visualization of the embedding.

68. The method of claim 64, further comprising evaluating a treatment based on at least a portion of the embedding.

69. The method of claim 67, further comprising evaluating a treatment based on at least a portion of the one or more UMAP plots.
